(19) United States
(12) Patent Application Publication
(10) Pub. No.: US 2013/0330378 A1
(43) Pub. Date: Dec. 12, 2013

(54) ANISOTROPIC BIOLOGICAL PACEMAKERS AND AV BYPASSES

(71) Applicants: Kevin Kit Parker, Waltham, MA (US); Crystal M. Ripplinger, Woodland, CA (US); Adam W. Feinberg, Pittsburgh, PA (US); Josue A. Goss, Somerville, MA (US); Patrick H. Campbell, Marlborough, MA (US)

(72) Inventors: Kevin Kit Parker, Waltham, MA (US); Crystal M. Ripplinger, Woodland, CA (US); Adam W. Feinberg, Pittsburgh, PA (US); Josue A. Goss, Somerville, MA (US); Patrick H. Campbell, Marlborough, MA (US)

(73) Assignee: PRESIDENT AND FELLOWS OF HARVARD COLLEGE, Cambridge, MA (US)

(21) Appl. No.: 13/878,383

(22) PCT Filed: Oct. 7, 2011
(86) PCT No.: PCT/US11/55398

§ 371 Date: Aug. 16, 2013

Related U.S. Application Data
(60) Provisional application No. 61/391,203, filed on Oct. 8, 2010.

(51) Int. Cl. A61L 31/16

(52) U.S. Cl. CPC A61L 31/16
USPC 424/400; 424/93.21; 435/180

(57) ABSTRACT

The present invention provides biological pacemakers or AV-node bypasses The biological pacemakers or AV-node bypasses of the invention are useful for the treatment of, inter alia, cardiac arrhythmias and AV-node conduction defects.

### RELATED APPLICATIONS

This application claims the benefit of and priority to U.S. Provisional Patent Application Ser. No. 61/391,203, filed on Oct. 8, 2010. The entire contents of this application are incorporated herein by reference.

### BACKGROUND OF THE INVENTION

Bradyarrhythmias—including sick sinus syndrome and atrioventricular block (AV block)—affect millions of people, and can result in hemodynamic collapse. Implantable artificial pacemakers are the standard of therapy for the treatment of bradyarrhythmia. However, such implantable devices are unresponsive to autonomic heart rate modulation, require invasive surgical implantation and replacement every 5-10 years, are susceptible to temporary malfunction in the presence of magnets (metal detectors or MRI machines) or environmental noise, and increase the patient's inflammatory response and risk of infection. Also, electronic pacemakers are often not suitable for pediatric patients, have a limited battery life, and long-term use can be associated with permanent cardiac tissue damage. Recent studies suggest that implantable cardiac device failure is a problem, with explants and device replacements due to failure averaging several hundred a year in the United States.

Biological pacemakers are one alternative to electrical pacing therapy. Biological pacemakers are responsive to autonomic modulation, require no external power source or replacement, present minimal inflammatory response, can be permanent, and can be autologous. Attempts at restoring cardiac automaticity with biologics have recently focused on two main approaches: gene therapy and cell transplantation. Gene-based approaches introduce genes directly into myocardial cells to restore or enhance automaticity, whereas cell transplantation approaches involve transplanting isolated spontaneously active or genetically-engineered cells directly into the myocardium. These transplanted cells must then electrically couple with the surrounding myocardium to effectively pace the heart. One of the central challenges of cell-based therapy is successful integration of transplanted cells within the three-dimensional architecture of the heart. In the absence of cues to direct their appropriate alignment with native heart tissue, isolated transplanted cells are unable to spatially align and effectively integrate into the existing three-dimensional architecture and are, thus, unable to provide improvement in functionality and generate an impulse to pace the heart.

Accordingly, there is a need for improved biological pacemakers or AV-node bypasses that can successfully establish connections with existing heart tissue and more closely replicate the function of a normal sinoatrial (SA) and/or atrioventricular (AV) node, thereby allowing more precise pacing control over the surrounding cardiac tissue.

### SUMMARY OF THE INVENTION

The present invention is based, at least in part, on the development of anisotropic muscle thin films (MTFs) that function as pacemakers and AV bypass nodes. Accordingly, described herein are methods and compositions for reconstructing the sinoatrial or atrioventricular nodal microarchitecture in vitro using tissue grafts that are easily implantable in vivo by minimally invasive means. These methods and compositions are applicable to both gene and cell therapies, but their applicability to cell-tissue applications will be described for the purposes of exemplification.

In one aspect, a biological pacemaker is provided that includes a flexible polymer layer and a population of pacemaker cells coated on the flexible polymer layer to form a tissue structure. In exemplary embodiments, the tissue structure is configured for epicardial or myocardial attachment and for the propagation of an action potential through the attached tissue.

To configure a tissue structure for epicardial or mycocardial attachment, the flexible polymer is patterned with, for example, essentially parallel lines of an extracellular matrix protein, e.g., fibronectin, that are spaced about 20 μM apart and are about 20 μM wide and about 2 μM high. The patterned flexible polymer (attached to the sacrificial polymer layer) is seeded with suitable cells and cultured to form an anisotropic tissue that will concatenate with a subject's heart, thereby forming gap junctions and is, thus, configured to propagate an action potential through the attached tissue to the subject's heart.

In other embodiments of the invention, a portion to substantially all of a portion at the site of placement of the epicardium, e.g., of the left or right atrium or the SA node, may be enzymatically digested to facilitate patch adhesion, concatenation of the patch to the subject's heart tissue, and propagation of an action potential through the attached tissue to the subject's heart.

In another aspect, the invention provides a method for fabricating a biological pacemaker by providing a base layer and coating it with a sacrificial polymer layer which, in turn, is coated with a flexible polymer layer that is more flexible then the base layer; seeding and culturing pacemaker cells to form a tissue structure; and releasing the flexible polymer layer with the tissue structure to produce a pacemaker graft. In exemplary embodiments, the graft is configured for epicardial or myocardial attachment and for the propagation of an action potential through the attached tissue.

To configure a tissue structure for epicardial or mycocardial attachment, the flexible polymer is patterned with, for example, essentially parallel lines of an extracellular matrix protein, e.g., fibronectin, that are spaced about 20 μM apart and are about 20 μM wide and about 2 μM high. The patterned flexible polymer (attached to the sacrificial polymer layer) is seeded with suitable cells and cultured to form an anisotropic tissue that will concatenate with a subject's heart, thereby forming gap junctions and is, thus, configured to propagate an action potential through the attached tissue to the subject's heart.

In yet another aspect, the invention provides a method of treating a patient with a bradyarrythmia, such as a bradyarrythmia caused by an SA node defect, by providing a biological pacemaker that includes a flexible polymer layer and a population of pacemaker cells coated on the flexible polymer layer to form a tissue structure, and attaching (e.g., by placing, suturing, and/or use of fibrin-based adhesives) the tissue structure to the patient's epicardium or myocardium. In some embodiments, the epicardial surface is treated, e.g., with a collagenase, to remove a portion of the epicardial surface at the site of attachment of the patch. In some exemplary embodiments, the biological pacemaker is configured for epicardial attachment and for the propagation of an action potential through the attached tissue to the remainder of the heart.

To configure a tissue structure for epicardial or mycocardial attachment, the flexible polymer is patterned with, for example, essentially parallel lines of an extracellular matrix protein, e.g., fibronectin, that are spaced about 20 μM apart and are about 20 μM wide and about 2 μM high. The patterned flexible polymer (attached to the sacrificial polymer layer) is seeded with suitable cells and cultured to form an anisotropic tissue that will concatenate with a subject's heart, thereby forming gap junctions and is, thus, configured to propagate an action potential through the attached tissue to the subject's heart.

In yet another aspect, the invention provides a method of treating a patient with AV nodal dysfunction or AV block by providing a biological AV bypass that includes a flexible polymer layer and a population of excitable cells coated on the flexible polymer layer to form a tissue structure which can bridge AV conduction defects and can propagate excitation from atria to ventricles with appropriate safety of conduction and a tunable AV delay. The tissue structure is attached (e.g., by placing, suturing, and/or use of fibrin-based adhesives) to the patient's ventricular myocardium. In some embodiments, the AV bypass is configured for myocardial or endocardial attachment and for propagation of an action potential through the attached tissue to the remainder of the heart.

To configure a tissue structure for endocardial or mycocardial attachment, the flexible polymer is patterned with, for example, essentially parallel lines of an extracellular matrix protein, e.g., fibronectin, that are spaced about 20 μM apart and are about 20 μM wide and about 2 μM high. The patterned flexible polymer (attached to the sacrificial polymer layer) is seeded with suitable cells and cultured to form an anisotropic tissue that will concatenate with a subject's heart, thereby forming gap junctions and is, thus, configured to propagate an action potential through the attached tissue to the subject's heart.

In some embodiments, the epicardial surface is treated, e.g., with a collagenase, to remove essentially all of the epicardial surface and at least a portion of the myocardium at the site of patch placement and expose at least a portion of the myocardium and/or endocardium. In some exemplary embodiments, the biological pacemaker is configured for myocardial or endocardial attachment and for the propagation of an action potential through the attached tissue to the remainder of the heart.

### BRIEF DESCRIPTION OF THE DRAWINGS

FIG. 1 is a schematic of the steps of one embodiment of the MTF fabrication process. (1) The substrates are fabricated on a glass cover slip spin coated with PIPAAm that provides temporary adhesion to a PDMS top layer. The PDMS is patterned with ECM, fibronectin (FN) in this case, to elicit cell adhesion and growth. (2) Substrates are placed in culture with a cell suspension to allow pacemaking cells to settle and adhere to the surface. (3) MTFs are cultured in an incubator until the pacemaking cells form a 2D tissue. (4) A desired shape is cut in the tissue/PDMS film using a scalpel. (5) The PIPAAm is dissolved by lowering the bath temperature below 35° C., releasing the MTF. The cutout shape floats free or is gently peeled off with tweezers. (6) The free-standing MTF is then used directly or modified further by folding into a 3D conformation.

FIGS. 2A-2F depict immunostained and phase-contrast images of cultured human Mesenchymal Stem Cells (hMSC) seeded at 2.5×10⁴ cells/cm² and stained on day 3. The left column shows immuno-stained images with medium gray, dark gray, and light gray corresponding to actin, fibronectin, and the nucleus, respectively. The right column shows phase-contrast images of the same tissue. A & B: Isotropic arrangement of cells; C & D: Anisotropic arrangement of cells, which are aligned horizontally; E & F hMSC arranged in horizontal lines.

FIG. 3 depicts spontaneous gap junction formation between cardiac myocytes cultured on a micropatterned substrate. Connexin 43 (white), sarcomere Z-lines are indicated by flourescent staining of a-actinin (gray), and nuclear DNA.

FIG. 4 depicts a magnified image of the edge of a MTF with cultured hMSCs. The hMSCs were seeded on thin films functionalized with fibronectin (20×20 μm 50 μg/ml lines w/2.5 μg/ml background) at a density of ˜250 k cells/well (25 k cells/cm2). On day 4 the media was allowed to cool down below 35° C. The film was cut with a razor blade inside the culture hood and pieces of the thin film were peeled off. Some of the pieces were placed in contact with myocyte monolayers and media was then added. Other pieces were removed and imaged.

FIG. 5 depicts immunostained and phase contrast images of hMSC-cardiomyocyte cultures. The constructs are comprised of alternating rows (20 μm wide) of hMSC and neonatal rat cardiomyocytes. Actin (white, left column), alpha-actinin (medium gray, right column and light gray, top row), and connexin-43 (medium gray, bottom column) are visualized in the hMSC-cardiomyocyte co-cultures. These images illustrate the concatenation and potential connectivity between the two cell types.

FIG. 6 depicts immunostained image of co-culture of hMSCs MTF and cardiomyocytes. The neonatal rat ventricular cardiomyocytes were seeded at a density of 1×10⁶ per 35 mm petri dish and the hMSC were seeded at a density of 1.50×10⁴ per petri dish on day 4 after myocyte seeding. The co-cultures were immunostained on day 7 with DAPI (dark gray), a-actinin (light gray) and Connexin-43 (medium gray) stains and overlaid. The cardiomyocytes were patterned in a non-confluent anisotropic mono-layer. White arrows point to the nuclei of an hMSC and a cardiomyocyte. The dashed circle points out the Cx-43 expressed inside an hMSC.

FIG. 7 depicts an immunostained image of an hMSC MTF and neonatal rat cardiomyocyte co-culture. The neonatal rat cardiomyocytes were seeded at a density of 1×10⁶ per well and the hMSCs were seeded at a density 1.50×10⁴ per well on day 4 after myocyte seeding. The co-culture construct was stained on day 7 with DAPI (dark gray), α-actinin (white), Cx43 (light gray), and actin (white) stains. The cardiomyocytes were patterned in a non-confluent anisotropic mono-layer. The inset focuses on the Cx-43 on the boundary between a cardiomyocyte and an hMSC.

FIGS. 8A-8D depict in vitro studies, in which an engineered anisotropic tissue (dark gray, myocyte nuclei indicated with medium gray (in A and B), gap junctions formed between cells (in B)) is cultured on a PDMS covered glass cover slip with a pacing MTF (wedge in A; top cells in B) attached to the apical surface. Gap junctions spontaneously form, electrically coupling the pacing MTF to the ventricular tissue for optical mapping experiments. C) Optical action potentials are recorded from an area of engineered cardiac tissue and display typical sharp upstrokes. D) Optical action potentials are recorded from an area with a pacing MTF attached and display slow diastolic depolarization due to the pacing current supplied by the MTF.

FIG. 9 is an image depicting a typical Langendorff working heart model apparatus. Such an apparatus was used to configure, optimize, and validate the attachment and function of the engineered MTF pacemakers ex vivo.

FIG. 10 is an image of exemplary pacing muscular thin film (MTF) patch comprised of a polymer base layer and aligned, patterned, and autonomously contracting cells configured for epicardial attachment. The patch was placed onto an adult rodent heart in a working heart model.

FIG. 11 is an image of an exemplary placement of a MTF patch configured for epicardial attachment. In this embodiment, the engineered patch was placed diagonally on the right atria of the adult rat heart. Although the film is transparent, it can be visualized by its reflection on the longitudinal right edge of the film.

FIG. 12 is an image showing enzymatic treatment of the epicardial surface of the right ventricle (RV) with a 1% collagenase solution to chemically digest the epicardial surface. Such a treatment was used to remove non-excitable cells to increase the pacemaking patch function and/or to chemically ablate the sinoatrial node (SA). The treatment took 1-15 minutes, followed by the addition of a buffered salt solution with 10% serum, which inactivates the digesting enzyme.

FIG. 13 depicts an exemplary electrocardiogram (ECG) from a Langendorff isolated working heart model following enzymatic digestion of the SA node and placement of a pacing MTF comprising ventricular myocytes. The microelectrode leads simulate a typical lead II patient placement. The anode was placed on the right atrium and the cathode on the ventricular apex, which measures the average depolarization of the ventricles from the apex to the atria.

FIG. 14 depicts a schematic of the cardiac conduction system and a pacing MTF architecture to replace the sinoatrial node (inset at left). The inset also depicts that a pacing MTF of the invention may comprise one or more cell types. For example, the conduction velocity of a biological bypass may be modulated by incorporating inexcitable cells such as cardiac fibroblasts or genetically modified excitable cells expressing specific gap junctions or ion channels.

FIG. 15 depicts an example of in vitro AV-Bypass MTF geometry. A) Culture of atrial and ventricular myocytes separated by an area of no cells. AV node-MTF is spanning the two cell populations. B) Depending on bridge geometry, unidirectional block may be achieved to prevent retrograde ventricular-to-atrial propagation. C) Same configuration as in (A) only area between cell populations is now filled with non-excitable cells such as cardiac fibroblasts which may lead to slowed conduction through the bridge.

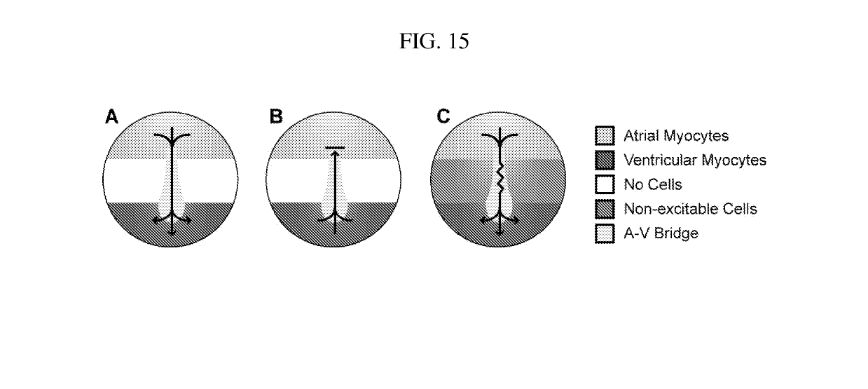

FIG. 16 depicts in vitro testing of a pacing MTF. A) Engineered myocardium with RH237 membrane stain on a 128 channel optical mapping system. The optical fiber array is depicted with a white circular outline for each photodiode. Scale bar is 100 μm. B). Action potential traces recorded for each photodiode. C) Activation map illustrates the arrival time of the action potential at points in the tissue. D) Isochrones mapping to the activation map are used to precisely calculate the action potential conduction velocity as it propagates thru the tissue. E) Time sequences show when the action potential arrives at different parts of the tissue. In these experiments, the tissue was paced by field stimulation. When a pacing MTF is fixed onto the tissue, these activation maps are used to determine if the pacing MTF is electrically controlling the whole tissue construct. A typical control experiment includes ablating, or removing, the pacing MTF and showing no ectopic activity from the same location and activation maps that were vastly different than when the pacing MTF was in place. Calculations of the conduction velocity from the arrival times in the isochrones is used to determine how well coupled, by gap junctions, the pacing MTF is to the myocardium. Local conduction velocity may be calculated from conduction velocity vector fields according to the method of Bayly et. al. (IEEE Trans Biomed Eng, 45(5):563-71, 1998).

FIG. 17 depicts action potential wavefront propagation in paced tissues with different anisotropy ratios (AR). In this example, all tissues were stimulated with a point electrode in the center of the tissue. The optical signals were normalized by the action potential amplitude to represent the transmembrane voltage in color. For each frame, the gray scale bar on the left indicates the resting state with dark gray and the peak of the action potential with medium gray. The white trace on the bottom is from a recording made at the site marked by the white square. The top panels show the action potential wavefront propagation in an isotropic tissue (AR=1). The middle and bottom panels show the wavefront propagation in anisotropic tissues with AR=2 and AR=3, respectively.

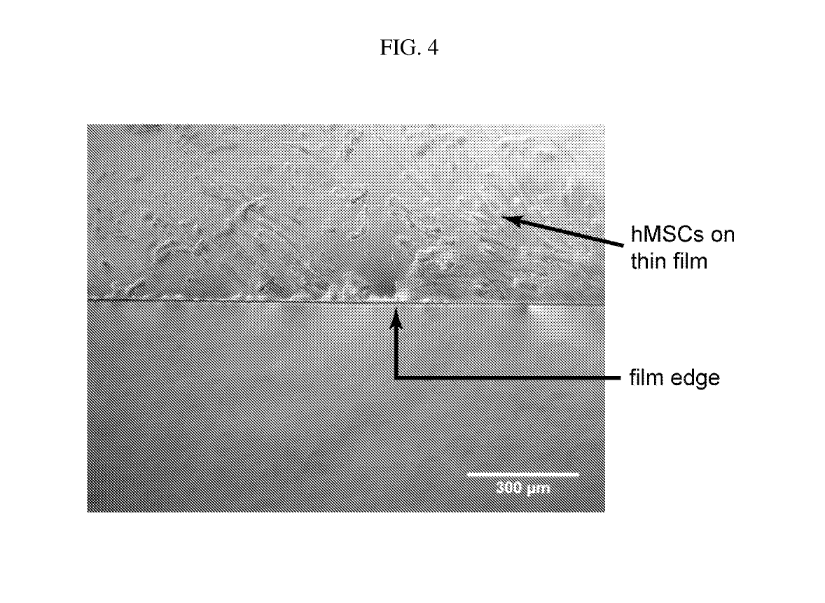

Normal cardiac muscle has anisotropic action potential propagation, which is required for coordination of the spatiotemporal contraction of the heart required for a sufficient ejection fraction of blood. Isotropic cardiac tissue lacks this uni-directional action potential propagation and thus, a heart composed of isotropic tissue is unable to pump sufficient blood to maintain systemic circulation. Anisotropy in the pacemaker MTF is required in order to properly couple with anisotropic cardiac muscle of the heart and to initiate action potential propagation in the appropriate direction. FIG. 4 illustrates the capability to orient cardiomyocytes uni-directionally and thus achieve anisotropic conduction in engineered cardiac tissue, this can be compared to the isotropic tissue where the action potential propagation is isotropic (i.e., circular wave front). The pacemaker MTF have cells oriented similarly using engineered surface chemistries.

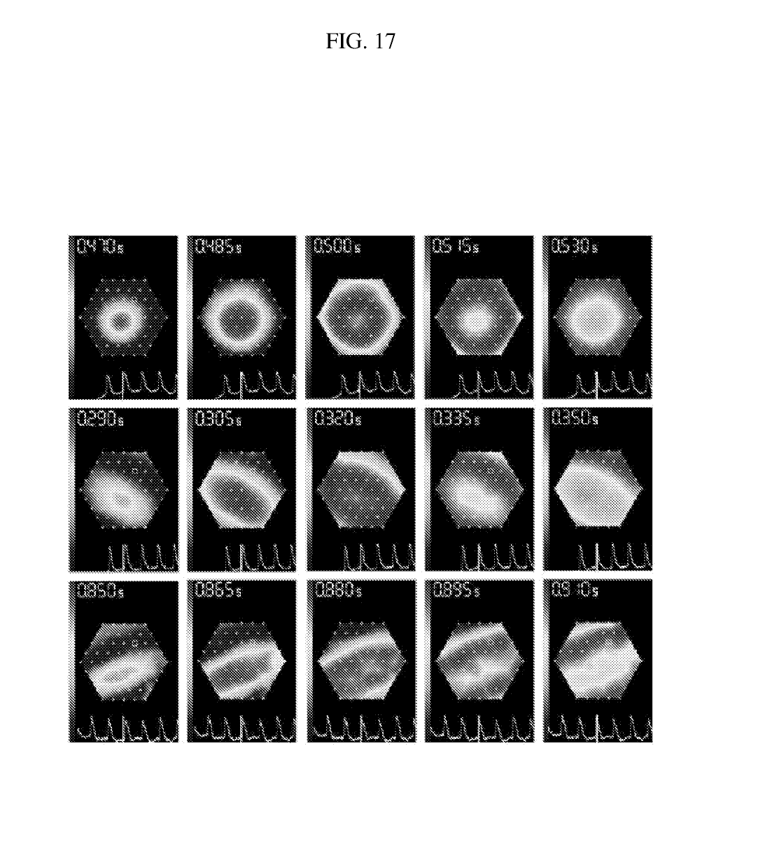

The anisotropy ratio (AR) is defined as the velocity of action potential propagation in the longitudinal direction divided by the transverse direction. The anisotropic engineered cardiac tissue in FIG. 17 has an anisotropy ratio ranging from 1-3. The AR also controls conduction velocity. In the case of the AV bypass, the AR of the cells on the bypass can be controlled to produce slower conduction (longer A-V delays) with lower ARs and faster conduction (shorter A-V delays) with higher ARs. For the biologic pacemaker, it is advantageous to make this anisotropic with the pacemaking cells aligned vertically from the superieror vena cava (SVC) to inferior vena cava (IVC). This cellular arrangement insulates the biologic pacemaker from the surrounding atrial myocytes by taking advantage of the native ‘block zone” (Bleeker et al., Circ Res 46(1): 11-22, 1980) and improving the safety of conduction.

The foregoing and other features and advantages of the invention will be apparent from the following, detailed description. In the accompanying drawings, like reference characters refer to the same or similar parts throughout the different views. The drawings are not necessarily to scale, emphasis instead being placed upon illustrating particular principles, discussed below.

### DETAILED DESCRIPTION

The present invention is based, at least in part, on the development of tissue constructs or anisotropic muscle thin films (MTFs) that function as biological pacemakers and AV bypass nodes. The anisotropic MTFs of the invention are fabricated on a biocompatible polymer patterned with extracellular matrix (ECM) substrates, e.g., fibronectin, laminin, collagens. The polymer/ECM scaffolds are incubated with a suspension of pacemaking cells, which adhere to the surface and form a 2-dimensional tissue of pacemaking myocardium, e.g., nodal pacemaking myocardium. The micropatterning of ECM substrates on the biocompatible polymer allows the cells to adhere to the polymer/ECM scaffold in an anisotropic arrangement that mimics the organization of myocardium in vivo. The cells comprising anisotropic MTFs are electrically coupled and are capable of transducing an action potential in vitro. When transplanted in vivo, anisotropic, pacing MTFs successfully pace native heart tissue and/or allow conduction between cell populations, thus functioning as a pacemaker or as an AV bypass.

### Anisotropic MTF-Based Pacemakers and AV-Node Bypasses

In one aspect, the present invention provides a biological pacemaker. In another aspect, the present invention provides a biological AV-node bypass. Such biological pacemakers and AV-node bypasses comprise an anisotropic muscle thin film (MTF) comprising a flexible polymer layer and a tissue structure comprising a population of cells coated on the flexible polymer layer. In one embodiment, the anisotropy of the MTF is configured to control the directionality of action potential propagation. In another embodiment, the anisotropy ratio of the MTF is configured to control the conduction velocity.

### (A) Polymer Scaffolds

Methods for fabricating a biological pacemaker functionalized with pacemaker cells and methods for fabricating a biological AV-node bypass functionalized with pacemaker cells are generally described in, for example, U.S. Patent Publication No. 2009/0317852, U.S. Provisional Patent Application Ser. No. 61/249,870, filed on Oct. 8, 2009, and PCT Publication No. WO 2010/127280, the entire contents of each of which are incorporated herein by reference, the entire contents of which are incorporated herein by reference.

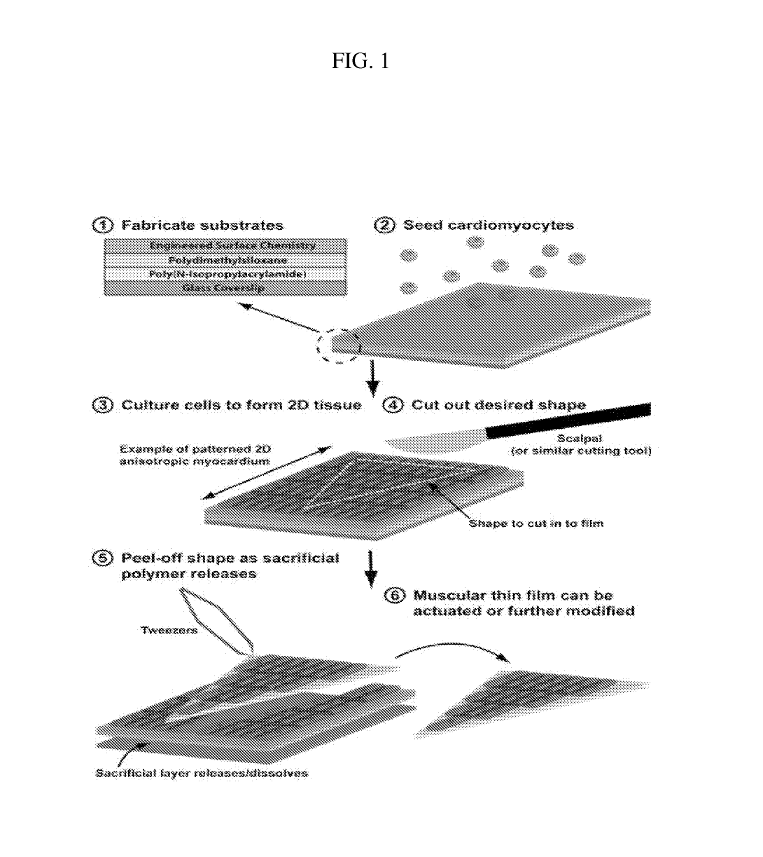

An exemplary embodiment of a method for fabricating a biological pacemaker functionalized with pacemaker cells and/or a biological AV-node bypass functionalized with pacemaker cells is depicted in FIG. 1.

The methods generally include, providing a base layer; depositing a sacrificial polymer on the base layer, thereby generating a sacrificial polymer layer; depositing a flexible polymer layer that is more flexible than the base layer on the sacrificial polymer layer; seeding cells on the flexible polymer layer; culturing the cells to form a tissue structure; and releasing the flexible polymer layer from the base layer.

The base layer used in the compositions and methods of the invention is formed of a rigid or semi-rigid material, such as a plastic, metal, ceramic, or a combination thereof. In particular embodiments, the Young's modulus of the base material used to form the base layer is greater than 1 mega-pascal (MPa). The base layer material may also be transparent, so as to facilitate observation. Examples of suitable base layer material include polymethylmethacrylate, polystyrene, polyethylene terephthalate film, silicon wafer, or gold. In one embodiment, the base layer is a silicon wafer, a glass cover slip, a multi-well plate or tissue culture plate.

The sacrificial polymer layer may be applied to the rigid base layer by “depositing” the sacrificial polymer onto the base layer. Depositing refers to a process of placing or applying an item or substance onto another item or substance (which may be identical to, similar to, or dissimilar to the first item or substance). Depositing may include, but is not limited to, methods of using spraying, dip casting, spin coating, or other methods to associate the items or substances. The term depositing includes applying the item or substance to substantially the entire surface as well as applying the item or substance to a portion of the surface.

In one embodiment, spin coating is used to deposit the sacrificial polymer layer to the base material. “Spin coating”, as used herein, refers to a process wherein the base layer is mounted to a chuck under vacuum and is rotated to spin the base layer about its axis of symmetry and a liquid or semi-liquid substance, e.g. a polymer, is dripped onto the base layer, with the centrifugal force generated by the spin causing the liquid or semi-liquid substance to spread substantially evenly across the surface of the base layer. The resulting sacrificial polymer layer serves to temporarily secure additional coatings that are subsequently formed thereon.

In one embodiment, the sacrificial polymer is a thermally sensitive polymer that is melted or dissolved to cause the release of the flexible polymer layer. An example of such a polymer is linear, non-cross-linked poly(N-Isopropylacrylamide), which is a solid when dehydrated, and which is a solid at about 37° C. (wherein the polymer is hydrated but relatively hydrophobic). However, when the temperature is dropped to about 35° C. to about 32° C. or less (where the polymer is hydrated but relatively hydrophilic), the polymer becomes a liquid, thereby releasing the patterned flexible polymer layer (Feinberg et al. (2007) Science 317:1366-1370).

In another embodiment, the sacrificial polymer becomes hydrophilic, thereby releasing hydrophobic coatings, with a change in temperature. For example, the sacrificial polymer can be hydrated, crosslinked N-Isopropylacrylamide, which is hydrophobic at about 37° C. and hydrophilic at about 35° C. or less (e.g., about 35° C. to about 32° C.).

In yet another embodiment, the sacrificial polymer is an electrically actuated polymer that becomes hydrophilic upon application of an electric potential to thereby release a hydrophobic structure coated thereon. Examples of such a polymer include poly(pyrrole)s, which are relatively hydrophobic when oxidized and hydrophilic when reduced. Other examples of polymers that can be electrically actuated include poly(acetylene)s, poly(thiophene)s, poly(aniline)s, poly(fluorene)s, poly(3-hexylthiophene), polynaphthalenes, poly(p-phenylene sulfide), and poly(para-phenylene vinylene)s.

In still another embodiment, the sacrificial polymer is a degradable biopolymer that can be dissolved to release a structure coated thereon. In one example, the polymer (e.g., polylactic acid, polyglycolic acid, poly(lactic-glycolic) acid copolymers, or nylons) undergoes time-dependent degradation by hydrolysis. In another example, the polymer undergoes time-dependent degradation by enzymatic action (e.g., fibrin degradation by plasmin, collagen degradation by collagenase, or fibronectin degradation by matrix metalloproteinase).

In yet still another embodiment, the sacrificial polymer is an ultra-hydrophobic polymer with a surface energy lower than the flexible polymer layer adhered to it. In this case, mild mechanical agitation will “pop” the patterned flexible polymer layer off. Examples of such a polymer include but are not limited to alkylsilanes (octadecyltrichlorosilane and isobutyltrimethoxysilane), fluoroalkylsilanes (tridecafluorotetrahydrooctyltrichlorosilane, trifluoropropyltrichlorosilane and heptadecafluorotetrahydrodecyltrichlorosilane), silicones (methylhydrosiloxane-dimethylsiloxane copolymer, hydride terminated polydimethylsiloxane, trimethylsiloxy terminated polydimethylsiloxane and diacetoxymethyl terminated polydimethylsiloxane), fluorinated polymers (polytetrafluoroethylene, perfluoroalkoxy and fluorinated ethylene propylene).

In an exemplary embodiment, the base material is a glass cover slip coated with a sacrificial polymer layer formed of linear poly(N-Isopropylacrylamide) (PIPAAm).

The sacrificial polymer layer provides temporary adhesion of the base material to a flexible polymer layer which can be likewise applied, e.g., via spin coating. Suitable polymers include, without limitation, any medical grade biocompatible flexible polymer. Examples of the elastomers that can be used to form the flexible polymer layer include polydimethylsiloxane (PDMS) and polyurethane. In other embodiments, thermoplastic or thermosetting polymers are used to form the flexible polymer layer. Alternative non-degradable polymers include polyurethanes, silicone-urethane copolymers, carbonate-urethane copolymers, polyisoprene, polybutadiene, copolymer of polystyrene and polybutadiene, chloroprene rubber, Polyacrylic rubber (ACM, ABR), Fluorosilicone Rubber (FVMQ), Fluoroelastomers, Perfluoroelastomers, Tetrafluoro ethylene/propylene rubbers (FEPM) and Ethylene vinyl acetate (EVA). In still other embodiments, biopolymers, such as collagens, elastins, and other extracellular matrix proteins, are used to form the flexible polymer layer. Suitable biodegradable elastomers include hydrogels, elastin-like peptides, polyhydroxyalkanoates and poly(glycerol-sebecate). Suitable non-elastomer, biodegrable polymers include polylactic acid, polyglycolic acid, poly lactic glycolic acid copolymers. In a preferred embodiment, the flexible polymer layer is a polydimethylsiloxane (PDMS) layer. For the case when the flexible polymer layer is PDMS, the thickness may be controlled by the viscosity of the prepolymer and by the spin coating speed, ranging from 14 to 60 μm thick after cure. After mixing the prepolymer, its viscosity begins to increase as the cross-link density increases. This change in viscosity between mixing (0 hours) and gelation (9 hours) is utilized to spin coat different thicknesses of flexible polymer layers. Alternatively the spin coating speed is increased to create thinner polymer layers. Following spin coating, the polymer scaffolds are either fully cured at room temperature (about 22° C.), or at 65° C.

The flexible polymer layer is then uniformly or selectively patterned with engineered surface chemistry to elicit (or inhibit) specific cell growth and function. The engineered surface chemistry can be provided via exposure to ultraviolet radiation or ozone or via acid or base wash or plasma treatment to increase the hydrophilicity of the surface. Additional suitable surface chemistries are provided in U.S. 2009/0317852, U.S. Provisional Patent Application Ser. No. 61/249,870, filed on Oct. 8, 2009, and WO 2010/127280, supra.

Pacemaker and AV-node bypass MTFs are generally patterned using the methods described in U.S. 2009/0317852, U.S. Provisional Patent Application Ser. No. 61/249,870, filed on Oct. 8, 2009, and WO 2010/127280, however the specific type of biopolymer used and geometric spacing of the patterning will vary with the application. For example, a specific biopolymer (or combination of biopolymers) may be selected to recruit, e.g., different integrins.

An engineered surface chemistry may be fabricated on the flexible polymer layer to enhance or inhibit cell and/or protein adhesion. In one embodiment, the engineered surface chemistry comprises a biopolymer, such as an extracellular matrix (ECM) protein. In one embodiment the ECM is a fibronectin. In another embodiment, the ECM is selected from the group consisting of laminin, a collagens, such as, Types I, IV, collagen, fibrin, and fibrinogen. The point of using these different ECM proteins and/or combinations there of is to recruit different integrin heterodimers, for patterning specific cell types.

In one embodiment, the ECM is not uniformly distributed on the surface of the flexible polymer, but rather is patterned spatially using techniques including, but not limited to, soft lithography, self assembly, vapor deposition, and photolithography.

“Biopolymer” refers to any proteins, carbohydrates, lipids, nucleic acids or combinations thereof, such as glycoproteins, glycolipids, or proteolipids.

Examples of suitable biopolymers that may be used for substrate functionalization include, without limitation:

(a) extracellular matrix proteins to direct cell adhesion and function (e.g., collagen, fibronectin, laminin, vitronectin, or polypeptides (containing, for example the well known -RGD- amino acid sequence));

(b) growth factors to direct specific cell type development cell (e.g., nerve growth factor, bone morphogenic proteins, or vascular endothelial growth factor);

(c) lipids, fatty acids and steroids (e.g., glycerides, non-glycerides, saturated and unsaturated fatty acids, cholesterol, corticosteroids, or sex steroids);

(d) sugars and other biologically active carbohydrates (e.g., monosaccharides, oligosaccharides, sucrose, glucose, or glycogen);

(e) combinations of carbohydrates, lipids and/or proteins, such as proteoglycans (protein cores with attached side chains of chondroitin sulfate, dermatan sulfate, heparin, heparan sulfate, and/or keratan sulfate); glycoproteins (selectins, immunoglobulins, hormones such as human chorionic gonadotropin, Alpha fetoprotein or Erythropoietin (EPO)); proteolipids (e.g., N-myristoylated, palmitoylated and prenylated proteins); and glycolipids (e.g., glycoglycerolipids, glycosphingolipids, or glycophosphatidylinositols);

(f) biologically derived homopolymers, such as polylactic and polyglycolic acids and poly-L-lysine;

(g) nucleic acids (e.g., DNA or RNA);

(h) hormones (e.g., anabolic steroids, sex hormones, insulin, or angiotensin);

(i) enzymes (e.g., oxidoreductases, transferases, hydrolases, lyases, isomerases, ligases; examples: trypsin, collegenases, or matrix metalloproteinases);

(j) pharmaceuticals (e.g., beta blockers, vasodilators, vasoconstrictors, pain relievers, gene therapy, viral vectors, or anti-inflammatories);

(k) cell surface ligands and receptors (e.g., integrins, selectins, or cadherins); and

(l) cytoskeletal filaments and/or motor proteins (e.g., intermediate filaments, microtubules, actin filaments, dynein, kinesin, or myosin).

In one embodiment of the invention, anisotropic cardiac tissue is engineered using alternating high density lines of ECM protein with either low density ECM protein or a chemical that prevents protein adhesion (e.g., Pluronics F127). The spacing of these lines as described previously (U.S. 2009/0317852; U.S. Provisional Patent Application Ser. No. 61/249,870, filed on Oct. 8, 2009; WO 2010/127280; Feinberg, (2007) Science 317:1366-1370), is typically 20 μm width at 20 μm spacing, however changing the width and spacing will change the alignment, thus changing the anistropy and thus changing the anisotropy ratio of the action potential propagation. The width and spacing of the ECM lines may be varied over the range from 100 nm up to 1000 μm, but typically the range is from 1 μm to 100 μm, and more specifically from 5 μm to 50 μm. The width and spacing of the ECM lines can be equivalent, or one can be larger than the other. For example, both the width and spacing can be 10 μm, or the width can be 5 μm and the spacing can be 20 μm, or conversely the width can be 20 μm and the spacing can be 5 μm. Typically the patterned ECM lines are parallel to one another. However they can also be at angles to one another ranging from 1° to 90°, but typically in the range from 5° to 45°. The purpose of altering the angle between the patterned lines of ECM protein is to control the directionality of action potential propagation, which of the example of the AV-bypass would allow conduction to be propagated from the atria to the ventricles, but not in the reverse direction. In addition to spacing and angle of the patterned ECM lines, the width of the MTF itself can be tapered to control directionality of action potential propagation. For example, a wide MTF strip that tapers to a narrow strip can propagate an action potential in that direction, but not in the opposite direction, which is once again key for creating an AV-bypass with uni-directional conduction.

MTFs can be specifically configured for epicardial attachment. As used herein term “configured for epicardial attachment” refers to construction of an appropriate size, shape and architecture such that the MTF can functionally attach to the epicardium. Such functional attachment includes the formation of adherens junctions and gap junctions between the cells of the MTF and the cells of epicardium to mechanically and electrically couple the MTF to the epicardium.

MTFs can also be specifically configured to propagate an action potential through the attached tissue. As used herein term “configured to propagate an action potential through the attached tissue” refers to construction of an MTF which is configured for epicardial attachment and that has the appropriate pattern of excitable cells to generate an electrical impulse suitable for inducing action potential through the tissue to which it is attached.

MTFs can be readily assessed to determine if they have been correctly configured for epicardial attachment such that they propagate an action potential through the attached tissue using optical mapping techniques known in the art and in vitro models of a working heart (see, e.g., the Examples set forth below).

Similarly, MTFs can be specifically configured for endocardial attachment. As used herein the term “configured for endocardial attachment” refers to construction of an appropriate size, shape and architecture such that the MTF can functionally attach to the endocardium. Such functional attachment includes the formation of adherens junctions and gap junctions between the cells of the MTF and the cells of endocardium to mechanically and electrically couple the MTF to the endocardium. The endocardial attached MTF is configured to propagate an action potential through the attached tissue. Assessment of MTF functional attachment to the endocardium is done using optical mapping techniques.

In other embodiments, a pacing MTF can be configured for myocardial attachment. As used herein term “configured for myocardial attachment” refers to construction of an appropriate size, shape and architecture such that the MTF can functionally attach to the myocardium. Such functional attachment includes the formation of adherens junctions and gap junctions between the cells of the MTF and the cells of myocardium to mechanically and electrically couple the MTF to the myocardium.

MTFs can also be specifically configured to propagate an action potential through the attached tissue. As used herein term “configured to propagate an action potential through the attached tissue” refers to construction of an MTF which is configured for myocardial attachment and that has the appropriate pattern of excitable cells to generate an electrical impulse suitable for inducing action potential through the tissue to which it is attached. Assessment of MTF functional attachment to the myocardium may be done using optical mapping techniques or other techniques well known in the art.

Pacemaking cells are seeded onto the flexible polymer layer, and are cultured to form a pacemaking tissue. A desired shape of the flexible polymer layer can then be cut, and the flexible film, including the polymer layer and tissue, can be removed from the sacrificial polymer layer. This releases the flexible polymer layer, producing a free-standing muscle thin film (MTF), composed of pacemaking cells.

### (B) Cells and Cell Culture

Electrically excitable cells suitable for use in biological pacemakers or AV-node bypasses include, but are not limited to, cells derived from a sinoatrial or an atrioventricular node, cells derived from the cardiac conduction system, ventricular myocardial cells, embryonic stem cells, induced pluripotent stem (iPS) cells, adult mesenchymal stem cells, adult cardiac resident stem cells, other adult stem cells (e.g., hematopoietic, fat), cardiac progenitor cells for the nodes and conduction system, or genetically engineered cells.

Suitable genetically engineered cells include, but are not limited to, any cell which has been genetically altered such that it possesses the electrical excitation or pacemaker properties necessary for biological pacemakers or AV-node bypass function. In some embodiments, cells are genetically engineered to express an ion channel that promotes pacemaking and/or electrical excitability. Suitable ion channels include, but are not limited to, hyperpolarisation-activated cyclic nucleotide-gated (HCN) channels, e.g., HCN1, HCN2, HCN3, or HCN4. Such ion channels are encoded by an HCN gene, e.g., a human HCN gene. Suitable adult mesenchymal stem cells expressing an HCN are described in WO2008011134 and Plotnikov et al., Circulation, 2007, 116(7):706-713, which are hereby incorporated by reference. In other embodiments, cells are genetically engineered to give them stem-cell characteristics such that they can be subsequently differentiated into a cell type which possesses the electrical excitation or pacemaker properties necessary for biological pacemaker or AV-node bypass function.

Stem cells for use in the compositions and methods of the present invention include embryonic (primary and cell lines), fetal (primary and cell lines), adult (primary and cell lines) and iPS (induced pluripotent stem cells).

The term “progenitor cell” is used herein to refer to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “progenitor cell” is used herein synonymously with “stem cell.”

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation”.

The term “embryonic stem cell” is used to refer to the pluripotent stem cells of the inner cell mass of the embryonic blastocyst (see U.S. Pat. Nos. 5,843,780, 6,200,806, the contents of which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells.

In one embodiment, progenitor cells suitable for use in the claimed methods are Committed Ventricular Progenitor (CVP) cells as described in PCT Application No. PCT/US09/060,224, entitled “Tissue Engineered Mycocardium and Methods of Productions and Uses Thereof”, filed Oct. 9, 2009, the entire contents of which are incorporated herein by reference.

Cells from any species can be used in the biological pacemakers and AV-node bypasses of the invention so long as they do not cause an adverse immune reaction in the recipient. In some embodiments, the excitable cells are syngeneic cells. In some embodiments, the excitable cells are human cells. In certain embodiments, the excitable cells are allogeneic cells. In other embodiments, the excitable cells are autologous cells.

To attach pacemaking cells to the flexible polymer layer, the flexible polymer layer is placed in culture with a cell suspension, and cells are allowed to settle and adhere to the surface. In the case of an adhesive surface treatment, cells bind to the material in a manner dictated by the surface chemistry. For patterned chemistry, cells respond to patterning in terms of growth and function. The seeding density of the pacemaking cells can be varied depending on the cell size and cell type. Suitable seeding densities include, but are not limited to, e.g., 1 to 10⁸ cells/cm²; 10 to 10⁷ cells/cm²; 10² to 10⁷ cells/cm²; 10³ to 10⁷ cells/cm²; 10⁴ to 10⁷ cells/cm²; 10⁵ to 10⁷ cells/cm²; or 10⁶ to 10⁷ cells/cm². In one embodiment, seeding densities can range from 1×10⁵ to 6×10⁵ cells/cm². In another embodiment, seeding densities are about 2.5×10⁴/cm².

The cell patterning of an MTF can be precisely controlled. In some embodiments, an MTF comprises a single continuous homogenous layer of excitable cells. In other embodiments, an MTF comprises multiple discrete regions of excitable cells. Suitable discrete regions include, without limitation, continuous fibers or threads. The width of such fibers or threads can be altered to control the amount of electrical conductivity of the MTF. MTFs comprising such continuous fibers or threads can be used, for example, to substitute for damaged Purkinje fibres in the ventricles.

The pacemaking cells on the substrates are cultured in an incubator under physiologic conditions (e.g., at 37° C.) until the cells form a two-dimensional (2D) tissue (i.e., a layer of cells that is less than 200 microns thick, or, in particular embodiments, less than 100 microns thick, or even just a monolayer of cells less than 15 microns thick). The anisotropy or isotropy of the tissue is determined by the engineered surface chemistry.

A specific shape (e.g., a triangle or oval or teardrop) can be cut in the flexible polymer film using a scalpel, punch, die, laser, or photolithography. The sacrificial layer is then dissolved or actuated to release the flexible polymer from the rigid base (e.g., by dropping the temperature below 35° C.); and the cut-out shape then floats free or is gently peeled off. In some embodiments, the pacemaking cells are aligned unidirectionally along the long axis of the pacemaker or AV-node bypass graft. The degree of cellular alignment, and thus anisotropy, can be precisely controlled and optimized for the shape and/or functional requirements of the graft by manipulating the engineered surface chemistry.

The provision of a directional, polarizing current can also be achieved by controlling the cellular architecture of the pacemaker graft. For example, a zone of non-excitable cells can be incorporated into one or more regions of pacemaker graft to effect a block of the polarizing current in a particular direction. By controlling the positioning of the non-excitable cells one can control the direction of the polarizing current produced by the pacemaker graft. Any non-excitable cells may be used to effect a block of the polarizing current. Such non-excitable cells include, but are not limited to, human cardiac fibroblasts, endothelial cells and vascular smooth muscle cells.

The flexible polymer layer is then uniformly or selectively patterned with engineered surface chemistry to elicit (or inhibit) specific cell growth and function. The engineered surface chemistry can be provided via exposure to ultraviolet radiation or ozone or via acid or base wash or plasma treatment to increase the hydrophilicity of the surface. Additional suitable surface chemistries are provided in U.S. 2009/0317852, U.S. Provisional Patent Application Ser. No. 61/249,870, filed on Oct. 8, 2009, and WO 2010/127280.

Pacemaker MTFs are patterned using the same basic methods as described previously for the cardiac MTFs (Feinberg et al. (2007) Science 317:1366-1370, U.S. 2009/0317852, U.S. Provisional Patent Application Ser. No. 61/249,870, filed on Oct. 8, 2009, and WO 2010/127280), but the specific type of ECM protein used and geometric spacing of the patterning will vary with the application. The proteins used will typically be fibronectin, laminin, collagens, e.g., Types I or IV, and fibrin (or fibrinogen). The point of using these different ECM proteins and/or combinations there of is to recruit different integrin heterodimers, which may be important for patterning specific cell types. For example, fibronectin is typically used for cardiomyocytes, but laminin and collagen can also be used.

Anisotropic pacing tissue is engineered using alternating high density lines of ECM protein with either low density ECM protein or a chemical that prevents protein adhesion (e.g., Pluronics F127). The spacing of these lines as described previously (Feinberg, 2007), is typically 20 μm width at 20 μm spacing, however changing the width and spacing will change the alignment, thus changing the anistropy and thus changing the anisotropy ratio of the action potential propagation. The width and spacing of the ECM lines may be varied over the range from 100 nm up to 1000 μm, but typically the range is from 1 μm to 100 μm, and more specifically from 5 μm to 50 μm. The width and spacing of the ECM lines can be equivalent, or one can be larger than the other. For example, both the width and spacing can be 10 μm, or the width can be 5 μm and the spacing can be 20 μm, or conversely the width can be 20 μm and the spacing can be 5 μm. Typically the patterned ECM lines are parallel to one another. However they can also be at angles to one another ranging from 1° to 90°, but typically in the range from 5° to 45°. The purpose of altering the angle between the patterned lines of ECM protein is to control the directionality of action potential propagation, which of the example of the AV-bypass would allow conduction to be propagated from the atria to the ventricles, but not in the reverse direction. In addition to spacing and angle of the patterned ECM lines, the width of the MTF itself can be tapered to control directionality of action potential propagation. For example, a wide MTF strip that tapers to a narrow strip can propagate an action potential in that direction, but not in the opposite direction, which is once again key for creating an AV-bypass with uni-directional conduction.

### (C) Biological Activity and In Vivo Delivery

As described above, certain embodiments of the invention allow for the formation of an a pacing MTF which is electrically coupled and capable of transducing an action potential in vitro and may be transplanted in vivo to successfully pace native heart tissue and/or allow conduction between cell populations, thus functioning as a pacemaker or as an AV bypass. Such a pacemaker may be used to treat a subject with a bradyarrythmia or a subject with an AV-node conduction defect.

To pace a heart, a pacemaker or AV-node bypass graft must be mechanically and electrically connected to the host cardiac tissue after implantation. When a pacing or AV-node bypass MTF is contacted with host tissue, cellular junctions are established, thereby connecting the MTFs with cells of the host tissue. Such junctions include gap junctions and adherens junctions. Accordingly, upon implanting the pacemaker graft in vivo, a temporary force is applied to hold the graft onto the host until these connections form. Cardiomyocyte MTF monolayers form conductive gap junctions within 30-45 minutes after physical contact is established between two cell monolayers. (Shimizu T, et al. (2006) J Biomed Mat Res 60(1):110-117; Haraguchi Y, et al. (2006) Biomaterials 2006; 27(27):4765-4774) Therefore, implanting the pacing or AV-node bypass MTFs in vivo will generally not require a special securing mechanism or suturing, however suturing and/or fibrin based surgical adhesives may be used. Any suitable means for accessing the heart tissue and implanting the pacemaker or AV-node bypass graft into the heart may be used including, but not limited to, e.g., thoracic surgery or transmyocardial catheter delivery. In some embodiments, a pacemaker or AV-node bypass graft is rolled up inside a transmyocardial catheter prior to implantation and subsequently unrolled when the site of implantation is reached. This site of implantation may be endocardial, myocardial, or epicarcardial depending on the specific pacing need of the heart and the underlying disease state that has necessitated pacing therapy. In certain embodiments, the epicardial surface of the site for attachment of the pacing MTF may be removed, e.g., chemically, at least in part, to facilitate coupling of the pacing MTF and the myocardium. A pacemaker MTF may be placed or attached to the right atrium or the left atrium. An AV-node bypass MTF may be placed or attached to the myocardium or endocardium of the left ventricle.

The exact size and shape of the MTF pacemaker or AV-node bypass is species- and patient-specific. For example, for in vivo testing in a rat heart, the MTF may only be approximately 10 mm² (e.g., 2-4 mm in length for a square or rectangular shape or approximately 3 mm in diameter for a circle). In some embodiments, the size and shape of the MTF pacemaker or AV-node bypass for in vivo testing is 1 mm², 2 mm², 3 mm², 4 mm², 5 mm², 6 mm², 7 mm², 8 mm², 9 mm², 10 mm², 15 mm², 20 mm², 25 mm², or 30 mm². For an adult human, the MTF is typically 2-4 cm in length for a square or rectangular shape or 3 cm in diameter for a circular shape. The size of the pacemaker graft can be designed according to the needs of the patient. Suitable surface areas for the pacemaker grafts include, but are not limited to, e.g., 1 to 10⁶ mm², 10 to 10⁵ mm², 10² to 10⁴ mm², or, 10² to 10³ mm²Suitable lengths for pacemaker grafts include, but are not limited to, e.g., 0.1, 05, 1, 2, 3, 4, 5, 6, 7, 8, 9, 10, 11, 12, 13, 14, 15, 16, 17, 18, 19, 20, 25, 30, 40, 50 or 100 cm. Patients with hypertrophic hearts may require larger pacemaker grafts than those with normal sized hearts. Likewise, pediatric patients may require smaller pacemaker grafts than adult patients.

The shape of the pacemaker or AV-node bypass MTF can be designed according to the needs of the patient. The overall shape of the MTF is optimized to possess desirable biological properties, and to efficiently deliver depolarizing current to the host myocardium with as few pacemaking cells as possible. For example, the shape of a pacemaker MTF can be designed to be elliptical, and thereby mimic the shape of a normal human SA node. Alternatively, the shape of the pacemaker or AV-node bypass graft can be specifically designed to deliver a directional, polarizing current to the surrounding cardiac tissue. Suitable shapes for delivering a directional, polarizing current including, but are not limited to, triangles, ovals or teardrop shapes. A triangle shape, for example, may allow for tuning the direction of wavefront propagation. The incorporation of non-excitable cells (cardiac fibroblasts, for example) may also be used to block propagation in one direction in order to deliver more depolarizing current in the opposite direction. This technique can increase the safety of conduction by mimicking or reinforcing the “block zone” region of the right atrium, an inexcitable region running between the superior and inferior vena cavae which is thought to prevent the atrial myocardium from loading the SA node.

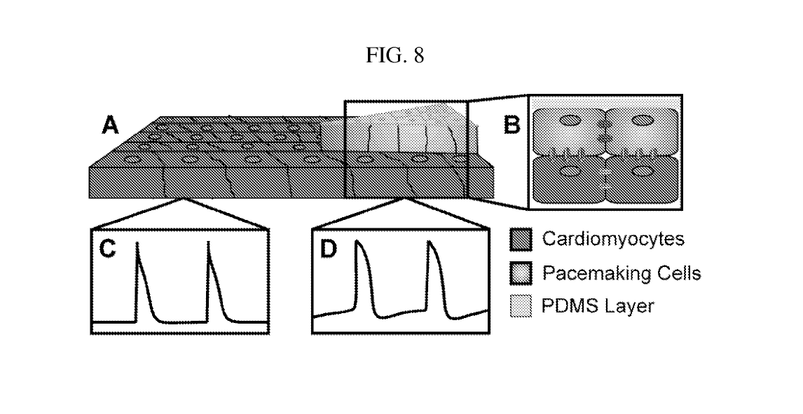

An AV-node bypass MTF is at least 0.5-10 cm in length (e.g., 0.5 cm, 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm, or 8 cm) to appropriately traverse the path from atria to ventricles in an adult human (either endocardially or epicardially depending on the transplant method). In a preferred embodiment, an AV-node bypass is at least 2-3 cm in length. An AV-node bypass can be shaped according to the needs of a patient, for example, shaped as a square, rectangle, triangle, or teardrop. In a preferred embodiment, an AV-node bypass is shaped as a teardrop. The teardrop shape allows safe delivery of current from atria to ventricles while preventing retrograde activation from ventricles to atria (see, for example, FIG. 8).

The present invention is next described by means of the following examples. However, the use of these and other examples anywhere in the specification is illustrative only, and in no way limits the scope and meaning of the invention or of any exemplified form. Likewise, the invention is not limited to any particular preferred embodiments described herein. Indeed, many modifications and variations of the invention may be apparent to those skilled in the art upon reading this specification, and can be made without departing from its spirit and scope. The contents of all references, patents and published patent applications cited throughout this application as well as the Figures are incorporated herein by reference.

### EXAMPLES

### Materials and Methods

### Cell Harvest and Cell Culture

All animal experiments were performed in accordance with the Harvard University Committee on Animal Care, which complies with United States Public Health Service standards and with other state and federal laws. Cardiac myocytes were dissociated from ventricles of 2 day old neonatal Sprague-Dawley rats using trypsin and collagenase and re-suspended in M199 culture medium supplemented with 10% heat-inactivated FBS, 10 mM HEPES, 3.5 g/L glucose, 2 mM L-glutamine, 2 mg/L vitamin B-12, and 50 U/mL penicillin. Isolated cells were differentially pre-plated in two 45 minute steps and re-suspended in culture medium. The standard bathing solution for electrophysiological studies contains 137 mM NaCl, 5.4 mM KCl, 1.2 mM MgCl₂, 1 mM CaCl₂, 20 mM HEPES (pH=7.4, warmed to 36° C. for experiments). For Ca⁺⁺ imaging, micropatterned myocytes are exposed to 5 μM fluo-3 AM (diluted from stock solutions containing 50 μg Fluo-3 AM, 25 μg Pluronic (Molecular Probes, Eugene, Oreg.) in 100 μL dimethyl sulfoxide) for 5 minutes followed by a 30 minutes wash in extracellular solution to allow time for deesterification.

Pacemaker cells are harvested in a similar fashion. Atrial myocytes are isolated from 2 day-old Sprague Dawley rats as described above. Excised right atrial tissue is agitated in a 0.1% trypsin solution cooled to 4° C. for approximately 14 hours. Trypsinized atria are dissociated into their cellular constituents via serial exposure to a 0.1% solution of collagenase type II at 37° C. for 2 minutes. The myocyte portion of the cell population is enriched by passing the dissociated cell solution through a nylon mesh with 40 μm pores, and then pre-plating twice for 45 minutes each time. Isolated myocytes are seeded onto muscular thin film substrates with patterned fibronectin matrices and grown in culture medium consisting of Medum 199 base supplemented with 10% heat-inactivated fetal bovine serum, 10 mM HEPES, 20 mM glucose, 2 mM L-glutamine, 1.5 μM vitamin B-12, and 50 U/ml penicillin. On the second day of culture, the serum concentration of the medium is reduced to 2%, and the medium is changed every 48 hours thereafter.

Human mesenchymal stem cells (hMSCs) were purchased from Lonza and cultured in MSC growing medium at 37° C. in a humidified atmosphere of 5% CO2. HMSCs were seeded onto MTF substrates patterned with fibronectin matrices at about 2.5×10⁴/cm² and cultured for three days.

### MTF Fabrication

PDMS thin film substrates were fabricated via a multi-step spin coating process. Glass cover slips (25 mm diameter) were cleaned by sonicating for 60 minutes in 95% ethanol and air dried. Next, poly(N-isopropylacrylamide) (PIPAAm, Polysciences) was dissolved at 10 wt % in 99.4% 1-butanol (w/v) and spin coated onto the glass cover slips for 1 minute at 6,000 RPM. Sylgard 184 (Dow Corning) polydimethylsiloxane (PDMS) elastomer was mixed at a 10:1 base to curing agent ratio and spin coated on top of the PIPAAm coated glass cover slip. Once mixed, the PDMS prepolymer slowly increases in viscosity reaching gelation at ˜8 hours. Thicker PDMS layers were formed by spin coating higher viscosity PDMS prepolymer between 0 and 6 hours post mixing allowing films from 14 to 60 μm thick to be formed. PDMS coated cover slips were then cured either at room temperature (˜22° C.) for 48 hours or at 65° C. for 4 hours. Different curing temperatures were used to control the curvature of the PDMS film when it is released from the cover slip upon dissolution of the PIPAAm layer.

### PDMS Surface Functionalization

The PDMS thin films were coated with either an isotropic or patterned layer of fibronectin (FN, Sigma). In either case, immediately prior to FN treatment the PDMS coated cover slips were oxidized using UV ozone (Model No. 342, Jetlight Company, Inc.) for 8 minutes to sterilize the surface and increase hydrophilicity for microcontact printing (μCP) (Tan et al., Tissue Eng 10(5-6): 865-72). Subsequent processing was performed in a biohood under sterile conditions. Isotropic FN was deposited by placing a 1 mL droplet of 25 μg/mL FN in sterile deionized (DI) water on the PDMS and incubating for 15 minutes. It is essential that water does not contact the periphery of the cover slip during this or any subsequent step because it would seep under the PDMS and prematurely dissolve the PIPAAm. Following FN incubation, excess protein was removed by washing 3 times with DI water and then air drying prior to cardiomyocyte seeding.

Anisotropic patterning of FN was performed using μCP. The basic μCP technique is well established and allows the rapid patterning of biomolecules on a variety of planar substrates using PDMS stamps. PDMS stamps were used to pattern alternating high and low density lines of FN on the PDMS coated glass cover slips in order to form anisotropic 2D myocardium, as based on previously published methods. PDMS stamps were fabricated with 20 μm wide, 2 μm tall ridges separated by 20 μm spacing. Briefly, silicon wafers were spin coated with SU-8 photoresist (Microchem) and exposed to UV light through a photomask selectively cross-linking regions of the photoresist. The photoresist was then developed and the non-exposed regions were removed. A negative of the patterned photoresist wafer was formed by casting PDMS prepolymer against it. Prior to each use, the PDMS stamps were sonicated in 50% ethanol for 30 minutes to sterilize and remove surface contaminants. Once dried, the PDMS stamp was inked with a 250 μL droplet of 50 μg/mL FN in DI water and incubated for 1 hour. The stamp was then rinsed twice in DI water to remove excess protein and dried under a stream of compressed air. High density FN lines were transferred from the stamp to the PDMS thin film by making conformal contact for 1 minute. Upon stamp removal a background surface chemistry was applied to the region in between the high density FN lines. To prevent cell adhesion in between the lines and create an array of discrete muscle fibers, a droplet of 1% Pluronics F127 (BASF Group) in DI water was spread over the patterned area and incubated on the PDMS surface for 15 minutes. To create anisotropic 2D myocardium low density FN lines in between the high density FN lines were used, where a droplet of 2.5 μg/mL FN in DI water was spread over the patterned area and incubated on the PDMS surface for 15 minutes. Following the incubation period, the PDMS film was washed 3 times with DI water, air dried and then seeded with pacemaking cells according to the protocol above.

### Immunostaining

Gap junctions and cadherins junctions were immunoflourescently detected as follows: Samples were first permeabilized in a cytoskeletal stabilizing buffer (300 mM sucrose, 100 mM NaCl, 3 mM MgCl₂, 0.5% TritonX100, 10 mM Pipes, pH 6), then fixed in 4% paraformaldehyde for 15 minutes and washed with PBS. To prevent nonspecific binding of secondary antibodies, a blocking procedure was used that includes incubation for 15 minutes in 5% serum from the species source of the secondary antibody, 1% BSA in PBS. The samples were then incubated with primary antibody to the desired target in PBS for 1 hour, washed, incubated in flourescently-labeled secondary antibody in PBS for 1 hour, and washed.

For histological examination, pacemaker constructs were placed in tissue embedding medium (Histo-Prep™, Fisher Scientific) and frozen at −80° C. Frozen samples were cryosectioned, mounted on Superfrost Plus glass slides (Fisher Scientific), and stored at −80° C. Immunohistochemical analysis of samples was conducted by immersing constructs in a solution of 4% paraformaldehyde and 0.5 μL/mL Triton X-100 for 15 minutes. Mouse monoclonal antibodies raised connexin 43 were used to label connexin channels between different cell types. Mouse monoclonal antibodies raised against connexin 40 may also be used to label connexin channels between ventricular myocytes and atrial pacemaker cells. Labeled proteins were visualized by applying goat anti-mouse IgG secondary antibodies conjugated to either Alexa Fluor 488 or Alexa Fluor 594.

Transfection of Green Fluorescent Proteins (gfp)

Transfections of atrial myocytes with gfp expression plasmids are accomplished with a component system formed by preincubation of Ad5dl312 adenovirus, poly-L-lysine. The expression plasmid is used to transfect cells that are cultured on micropatterned islands as described above. Fluorescent microscopy is used to verify transfection efficiency. Transient transfection of gfp- and yfps, such as gfp-paxillin, is accomplished using Effectene transfection reagent (Qiagen, Chatsworth, Calif.).

### Optical Mapping of 2-D Engineered Cardiac Cells

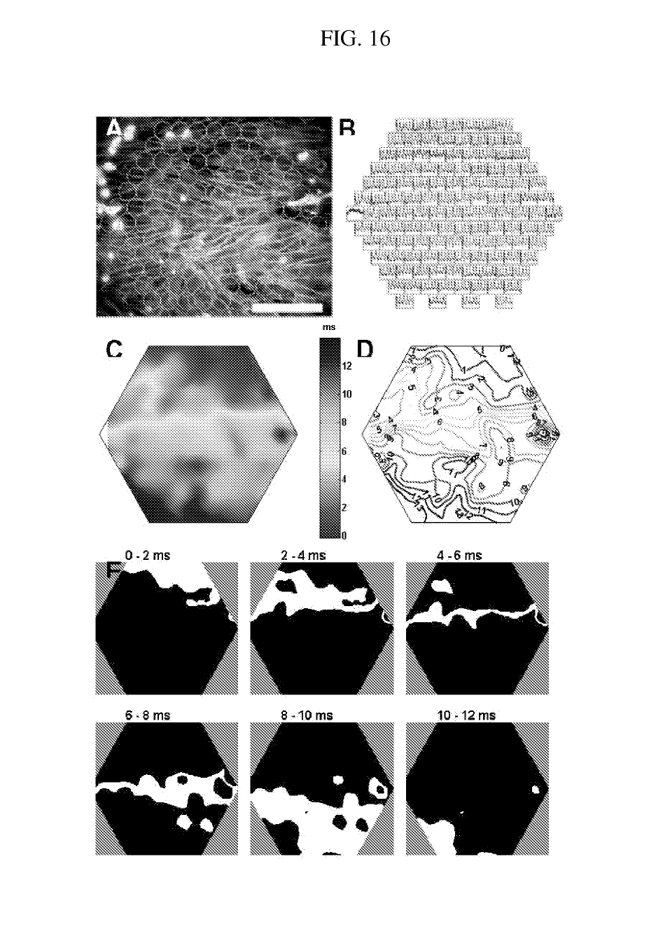

The optical mapping system (OMS) is a high-speed, high-sensitivity 124-channel photodiode system that is optimized for dynamic fluorescence imaging of voltage-sensitive and calcium-sensitive dyes (FIG. 16). The OMS consists of 124 independent optical fibers arranged in a honeycomb array, connected through the baseport of an inverted microscope. Each fiber is connected to a discrete photodiode transimpedance amplifier. The current through each photodiode is amplified by a 100 MΩ/A transimpedance gain, AC-coupled, and scaled by a non-inverting gain of 10 V/V prior to discretization by a 12-bit A/D converter. Signal bandwidth is hardware-limited to 2.5 kHz to minimize front-end noise while providing adequate bandwidth to detect action potentials. Maximum spatial resolution is 10 μm. Maximum sample rate is 5 kHz (200 μs) when all pixels are recorded, and can be increased up to 200 kHz (50 μs) when a subset of pixels is recorded. Fluorescence signals from each optical fiber are low-pass filtered at 100 Hz, normalized, and dVₘ/dt is calculated by a 5-point numerical derivative. Activation times are determined by dVₘ/dtₘₐₓ. Conduction velocity vector fields are calculated from activation maps (FIG. 17).

Optical recordings of transmembrane potential (Vₘ) are performed in Tyrode's solution of the following composition (in mM): NaCl 135.0, CaCl₂ 1.8, KCl 5.4, MgCl₂ 1.0, NaH₂PO₄ 0.33, HEPES 5.0 and glucose 5.0. The excitation-contraction uncoupler, Blebbistatin (10 μM, Calbiochem), is added to the solution to reduce motion artifacts. The pH is adjusted to 7.4 and the temperature maintained at 35° C.

Fluorescence recordings are obtained with the voltage sensitive dye RH237 (Invitrogen). A 2 mM stock solution of RH237 in dimethyl sulfoxide (Sigma) is prepared and stored at 4° C. The stock solution is diluted in Tyrode's solution to a final concentration of 8 μM. Cell cultures are incubated in the dye solution for 5 minutes, washed 3 times with Tyrode's solution, and incubated in Tyrode's solution containing Blebbistatin for 10 minutes before imaging. Using an inverted microscope (Zeiss Axiovert 200) with a 40× objective (Zeiss EC Plan-NEOFLUAR, numerical aperture 1.3), fluorescence recordings are obtained. Cell cultures are exposed for 1-2 sec to excitation light (530-585 nm). Emitted light is longpass filtered at 615 nm and focused onto the hexagonal array of 124 optical fibers each coupled to a photodiode. At 40×, each optical fiber corresponds to a 25 μm-diameter tissue area. The photocurrent from each diode is converted to a voltage, amplified and digitized at 12-bit resolution at a sampling rate of 5 kHz.

### Optical Mapping of Isolated Rat Hearts

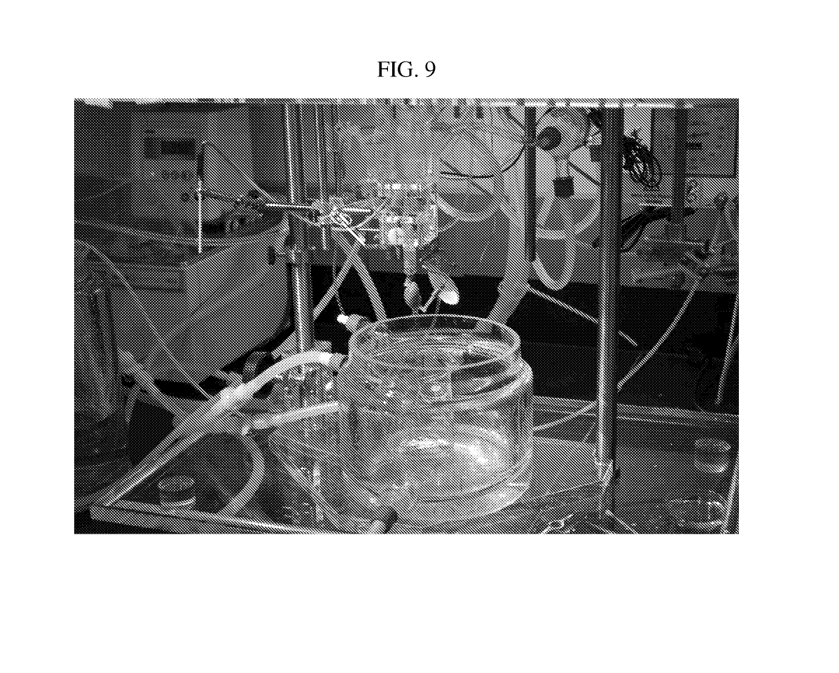

After intraperitoneal (IP) injection of 300 units heparin, rats were anesthetized with sodium pentobarbital (50 mg/kg IP). Once surgical-depth anesthesia is reached, hearts were quickly excised via a midsternal incision. Hearts were placed on a Langendorff apparatus and retrogradely perfused through the aorta with warm (36° C.), oxygenated (95% O₂, 5% CO₂) modified Tyrode's solution of the following composition (in mM): NaCl 128.2, CaCl₂ 1.3, KCl 4.7, MgCl₂ 1.05, NaH₂PO₄ 1.19, NaHCO₃ 20 and glucose 11.1 (FIG. 9). The pH was maintained at 7.4 by adjusting the CO₂. The perfusion rate was adjusted to maintain an aortic pressure of 60-70 mmHg The excitation-contraction uncoupler, Blebbistatin (10 μM, Calbiochem, La Jolla, Calif.), is added to the perfusate to eliminate motion artifacts in the optical recordings caused by muscle contraction. The heart is then stained with the voltage-sensitive dye di-4-ANEPPS (5 minutes, 1.3 μM in the perfusate). Optical action potentials are recorded at high spatial resolution using a MiCAM Ultima-L CMOS camera (0.1 ms, 100×100 pixels). Optical fluorescence signals (F) are recorded from a region of approximately 30×30 mm with a spatial resolution of 300 μm at a rate of 1000-5000 frames/s. The signals are low-pass filtered, differentiated (dF/dt), normalized, plotted as two-dimensional intensity graphs, and overlapped as frames with the image of the preparation to produce animations.

### Removal of Epicardial Tissue to Improve Patch Adhesion & Connectivity

In some cases it may be advantageous to remove at least a portion of the epicardial layer of the heart to allow direct pacing cell to myocardial contact. This can be done surgically via physical scraping or peeling with traditional surgical tools.

Another (more gentle) approach is an enzymatic digestion involving isolating the region of interest, avoiding digesting non-target areas of the heart and surrounding tissues by, e.g., placing a tubular structure, such as a silicone ring, around the area of interest on the heart in order to prevent unwanted digestion of neighboring tissue. This structure can have an outer diameter (OD) of 1 mm to 100 mm (a typical OD would be 15 mm) and a wall thickness of 0.5 mm to 10 mm The structure can be made out of silicone or any other elastomeric, biocompatible polymer.

An additional method to avoid digesting non-target areas of the heart is local application of the enzyme using an absorbent material, such as a gauze or cotton-tipped applicator. Suctin was applied to a region of interest on the heart, and the area was perfused with the enzyme followed by the neutralizing solution. The area of suction is similar to the structure in the approach above, but instead of an open system, it was closed to the environment, allowing for a negative pressure which would allow for better isolation of the area of interest. A negative pressure was applied that was sufficient to mitigate enzyme leakage but not enough to disrupt blood flow to the heart. Suitable pressures range from 760 Torr, to no less than 500 Torr. Once the area was isolated, the system was perfused with digesting and neutralizing solutions.

Two enzymes are particularly relevant for cardiac epicardial digestion: collagenase (type I, type II, type III, and type IV) to remove collagen extracellular matrix and trypsin, a non-specific serine protease. Additionally, both of these enzymes can be neutralized. Other enzymes that might be used include papain, elastase, hyaluronidase, and dispase. One % to 50% solutions of enzyme in an isotonic salt solution at 50 degrees Fahrenheit to 101 degrees Fahrenheit, e.g., about 98 degrees Fahrenheit, for 10 secondsw to 30 minutes, e.g, about 2-10 minutes may be used.

In order to neutralize enzymatic digestion of the epicardium and avoid both over-digesting the cardiac tissue and potentially destroying the pace making patch, about a 10% serum solution, either from the patient's own serum or from commercially-purchased sources was added. Serum solution for neutralization may be at a concentration of about 1-75% in an aqueous buffer, such as a phosphate-buffered saline, isotonic saline solution, or a lactated ringer's solution. If trypsin is used as the enzyme, a soybean-based trypsin inhibitor can be added to neutralize the reaction.

### Example 1

### In Vitro Construction of a Pacing Muscular Thin Film

To demonstrate the construction of pacing MTFs, fibronectin was micropatterned onto PDMS coated glass coverslips and seeded with either ventricular myocytes or human mesenchymal stem cells (hMSCs). HMSCs are stable in cell lines and have low antigenicity. They are also able to transfer dye and to transmit current to one another, to other cell lines, and to myocytes (Potapova I., et al., (2004) Circ. Res 94:952-959; Valiunas V., et al. (2004) J. Physiol 555.3:617-626). Moreover, adult human mesenchymal stem cells form Cx43 junctions among themselves and with ventricular myocytes.

Specifically, linear patterns of 20 μm wide lines of fibronectin were transferred onto UV-Ozone treated PDMS (silicone polymer) coated coverslips. Unprinted areas were then blocked with Pluronics-F127 surfactant to prevent cell adhesion. For the construction of MYFs with anisotropic patterns, the same 20 μm wide lines of fibronectin were printed and then immersed in a 2.5 μg/ml fibronectin solution to provide a low background concentration of fibronectin. Isotropic fibronectin was created by coating the coverslips with a 25 μg/ml fibronectin solution. Following fibronectin patterning, cells, such as hMSCs, were seeded onto the substrates at a density of 1,000-250,000 cells/cm² and allowed to attach and proliferate in appropriate culture medium. After 3 days in culture, the thin films were separated from the glass and manipulated using surgical forceps. The pacing MTFs were fixed and stained for actin (phalloidin: medium gray), fibronectin (anti-fibronectin: dark gray), and the nucleus (DAPI: light gray).

Tissue engineered pacemakers may also be made by harvesting the sinoatrial node from neonatal rat right atria, chemically dissociating the cells, and culturing them on micropatterned MTFs. More specifically, the right atria from neonate rats is harvested, carefully dissected, and those myocytes in the region of the sinoatrial node are chemically dissociated. These myocytes are cultured on micropatterned MTFs to form an anisotropic tissue structure with autonomous beating capability

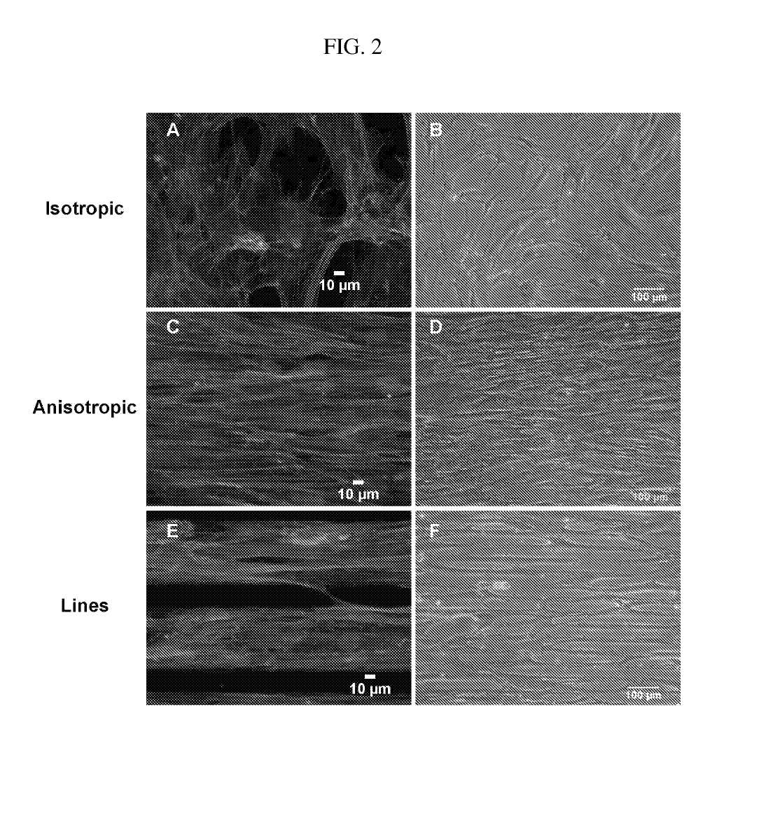

FIG. 2 shows that the cells of a pacing MTFs comprising hMSCs arrange themselves with the patterns created.

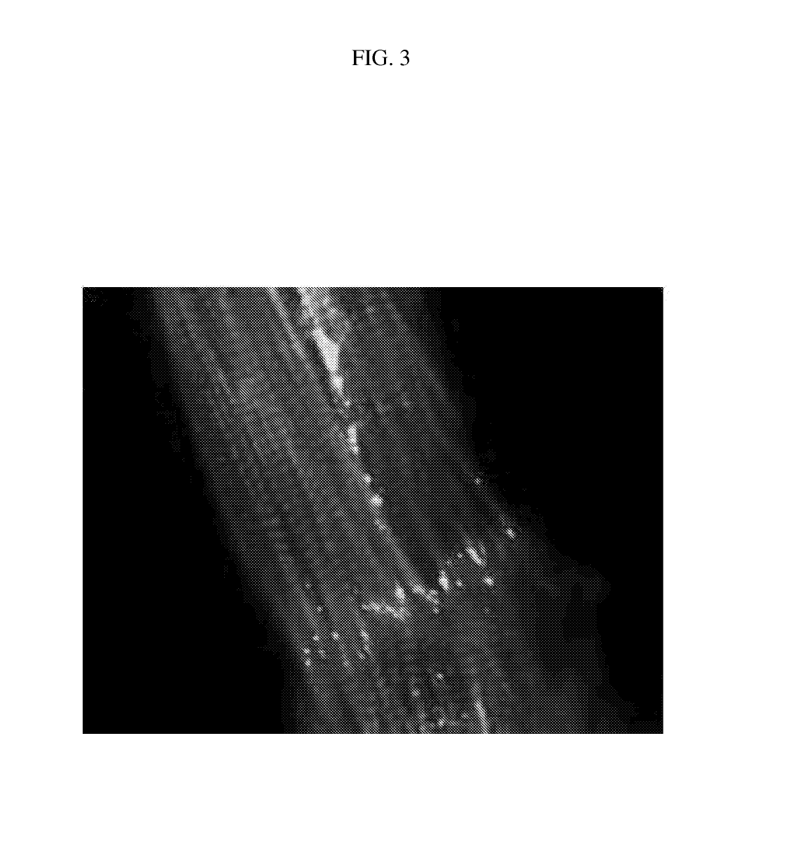

FIG. 3 shows that cells of a pacing MTF comprising cardiac myocytes spontaneously form gap junctions between cardiac myocytes.

FIG. 4 shows an image of the edge of a pacing MTF. The hMSCs were seeded on thin films functionalized with fibronectin (20×20 μm 50 μg/ml lines w/2.5 μg/ml background) at a density of ˜250,000 cells/well (25,000 cells/cm²). On day 4 the media was allowed to cool down below 35° C. The film was cut with a razor blade inside the culture hood and pieces of the thin film were peeled off. Some of the pieces were placed in contact with myocyte monolayers and media was then added. Other pieces were removed, immunostained, and imaged.

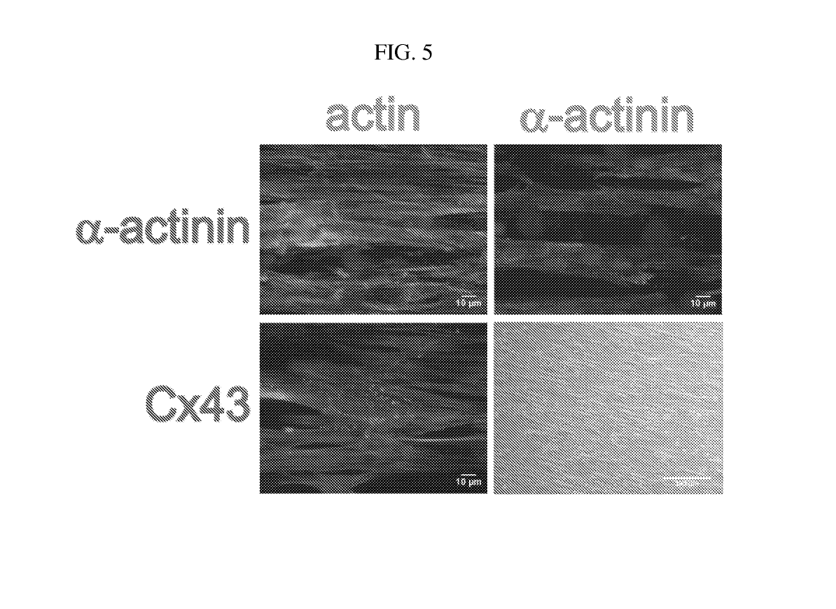

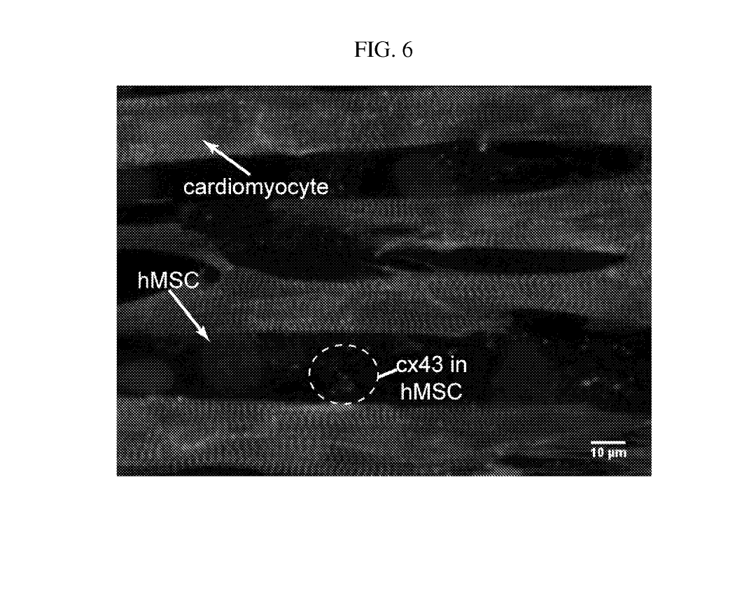

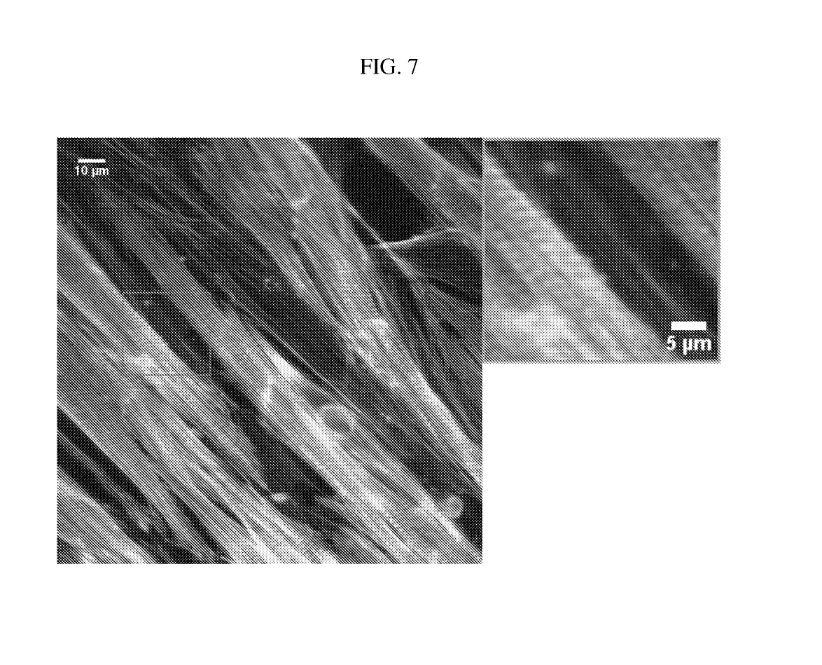

In order to demonstrate that the pacing MTFs comprising cultured hMSCs can form cell to cell connections (concatenate) and couple with cardiomyocytes in vitro, an MTF comprising cultured hMSCs was co-cultured with a monolayer of neonatal rat cardiomyocytes. The immunostained and phase contrast images shown in FIGS. 5-7 demonstrate that the two cell types concatenate and form Cx43 gap junctions.

To demonstrate that the tissue engineered pacemaker is capable of pacing control of cardiac tissue in vitro, immunohistochemical analysis is also performed on a pacing MTF placed on and attached to engineered ventricular myocardium (FIG. 8). Staining is used to demonstrate the formation of gap junctions between the ventricular myocardium and the pacemaking cells. It has been shown that ventricular myocytes predominantly express Cx43 gap junctions and that atrial myocytes primarily express Cx40 gap junctions and that these proteins will form a conductive heterotypic gap junction that will support propagation of an action potential between two myocytes. Therefore, after staining for both of these proteins, confocal microscopy is used to demonstrate that the gfp-expressing atrial myocytes are electrically coupling to the ventricular myocytes of the larger engineered tissue. Furthermore, immunostaining for cadherins is also used to show the formation of junctions between the atrial and ventricular myocytes.

Furthermore, to demonstrate functional coupling of the pacemaker to the tissue, physiological experiments with the optical mapping system described above are conducted to spatially map action potential propagation. Pacing control of the engineered myocardium with the pacing MTF is further demonstrated by using channel blockers against leaky Na⁺ ion channels which drive the autonomous pacing capability of the pacemaking cells. The efficacy of the pacemaker is shown by doing wash in, wash out of the channel blockers in conjunction with optical mapping.

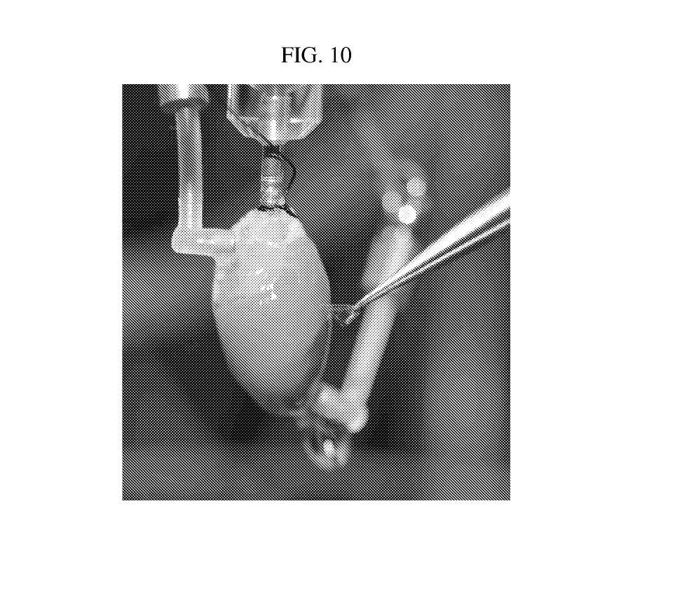

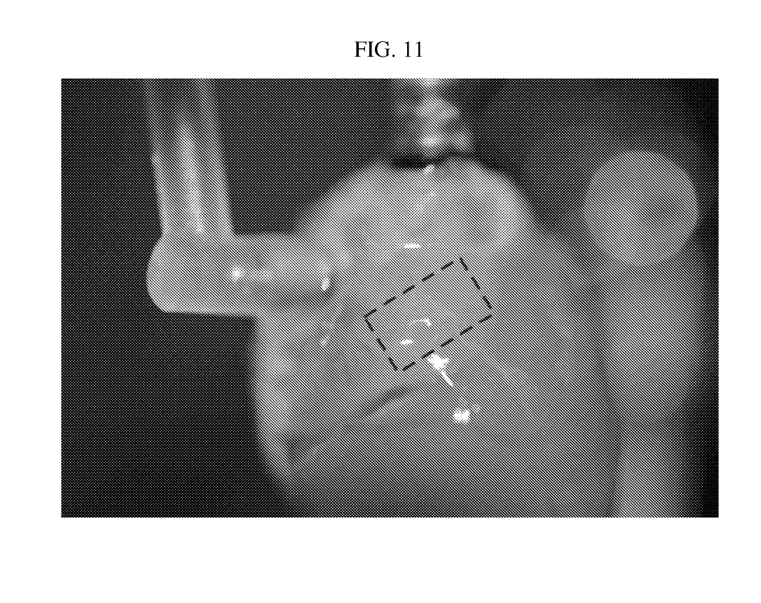

Using a Langendorff working heart model, pacing of an engineered pacing MTF was demonstrated (see, e.g., FIG. 9). As depicted in FIG. 10, an MTF patch comprising of polymer base layer and aligned, patterned, and autonomously contracting cells cultured from hMSCs and configured for epicardial attachment was placed onto an adult rodent heart in a Langendorff working heart model. The engineered patch was placed diagonally on the right atria of the adult rat heart (see FIG. 10), or the engineered patch is placed diagonally on the left atria of the adult rat heart (see FIG. 11).

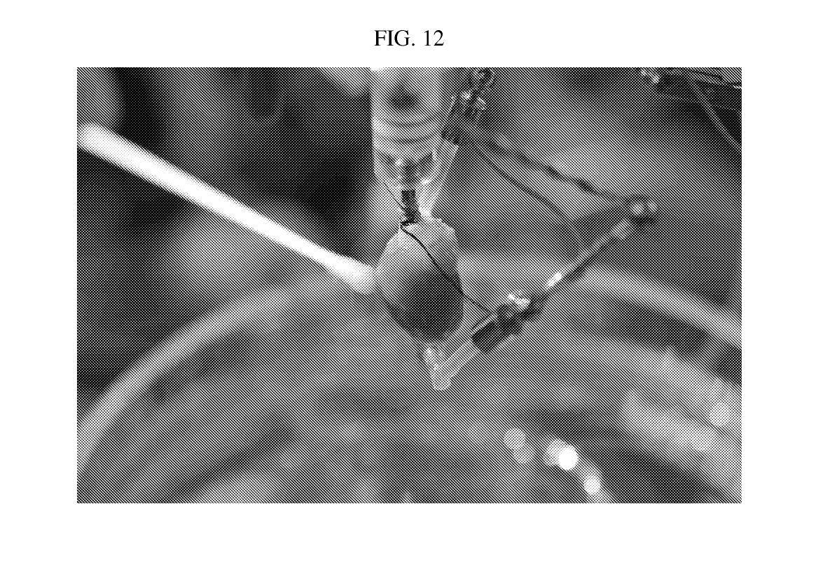

Furthermore, to remove non-excitable cells to increase the pacemaking patch function and/or to chemically ablate the sinoatrial node (SA), the epicardial surface of the right ventricle (RV) was treated with a 1% collagenase solution to chemically digest a portion or essentially all of the epicardial surface. The treatment was effected for about 1-15 minutes, followed by the addition of a buffered salt solution with 10% serum, which inactivates the digesting enzyme (see FIG. 12).

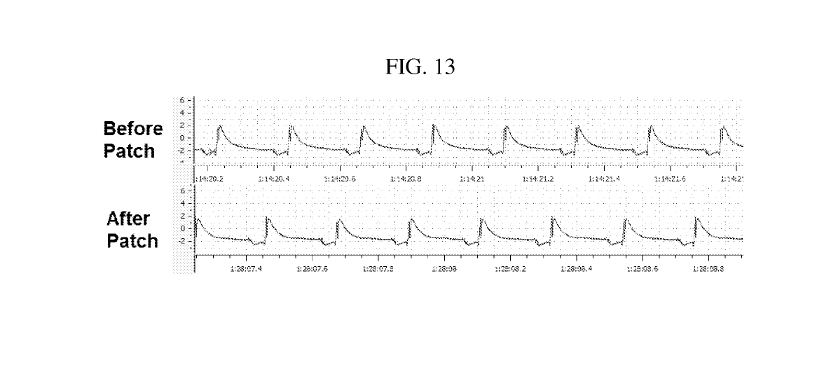

FIG. 13 depicts an exemplary electrocardiogram (ECG) data from a Langendorff isolated working heart model following enzymatic digestion of the SA node and placement of a pacing MTF comprising ventricular myocytes. The microelectrode leads simulate a typical lead II patient placement. The anode was placed on the right atrium and the cathode on the ventricular apex, which measures the average depolarization of the ventricles from the apex to the atria.

### Example 2

### Surgical Implantation of Pacing Muscular Thin Films

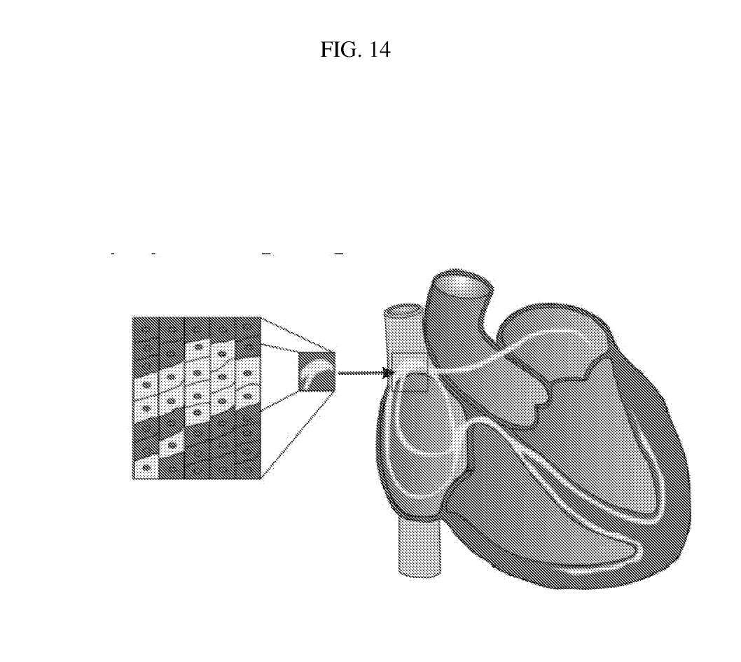

Surgical implantation of pacing MTFs is accomplished by surgically or enzymatically ablating the sinoatrial node in anesthetized rats and placing pacing MTFs on the apical surface of the right atria (see FIG. 14). More specifically, surgical ablation of the sinoatrial node is accomplished by cauterizing the node in vivo during survival surgery (Tamayski et al., Physiol Genomics. 2004: 16(3) pp. 349-60). Pacing is restored by implantation of a pacing MTF constructed as described in Example 1. Briefly, 70 mg/kg of pentobarbital sodium is administered to induce anesthesia. After an adequate depth of anesthesia is attained, the rodent is placed in a supine position and a taut 5-0 ligature is situated behind the front upper incisors to keep the neck slightly extended. The tongue is retracted and held with forceps while inserting a 20 gauge catheter into the trachea. The catheter is then attached to a ventilator via a Y-shaped connector. Ventilation is performed using a tidal volume of 200 uL and a respiratory rate of 133/min with 100% oxygen provided to the inflow of the ventilator. Prior to incision, the chest is disinfected with betadine solution, 70% ethyl alcohol, and 0.1 mL of 0.1% lidocaine introduced under the skin. The chest cavity is opened by an incision 1 to 2 mm above the left armpit and a chest retractor is applied to allow visualization of the heart. The pericardial sac is opened and pulled apart, the right atria is identified and its apical surface burned with a cauterizing electrode. When atrial contractions cease, a previously prepared pacing MTF is sewn onto the atrial surface with a 7-0 silk suture. Finally, the lungs are over-inflated, and the chest cavity, muscles and skin are closed layer by layer with 6-0 nylon and 6-0 absorbable (for muscles) sutures. The duration of the whole procedure is approximately 15-20 min.

Hearts of surviving rats are harvested for optical mapping studies. If the pacing MTF is successfully implanted, the heart will have a unique activation sequence with the earliest activation arising from the location of the pacemaking MTF. This activation sequence will not be replicated in control experiments accomplished by pacing the heart at other locations. Furthermore, the right atria with the pacing MTF attached is harvested for in vitro optical mapping experiments and postmortem histology. Immunostaining is done to demonstrate the formation of gap junctions from Cx 40, 43, and 45, the formation of adherens junctions, as well as to mark localized angiogenesis, fibrosis, and neural innervation. Additionally, myocytes on the pacing MTF are transfected with gfp prior to implantation and fluorescent microscopic examination of the right atria post mortem is used to determine if any of these cells migrated away from the graft site.

### Example 3

### Constructing Atrioventricular Muscular Thin Films

Engineered atrioventricular muscular thin films (AVN-MTFs) are constructed in a similar manner as the pacing MTFs. The AVN-MTF includes a flexible polymer layer and a population of excitable cells (e.g., cells derived from a sinoatrial or atrioventricular node, atrial or ventricular myocytes, embryonic stem cells, adult mesenchymal stem cells, or genetically engineered cells) coated on the flexible polymer layer to form a tissue structure which can bridge AV conduction defects in vitro. The biologic AVN-MTF will bridge conduction obstacles with an optimal A-V delay and unidirectional conduction block to prevent retrograde V-A activation. To test the properties of the AVN-MTF in vitro, AVN-MTFs will be transplanted onto populations of atrial and ventricular myocytes separated by an obstacle (FIG. 13). Optical mapping will be used to confirm conduction between the two cell populations.

Several design parameters will be varied to achieve optimal conduction properties of the AVN-MTF. The degree of anisotropy of the excitable cells on the AVN-MTF as well as the width and length of the MTF will be varied to determine the range of A-V delays that can be achieved. As an alternative means of modulating the A-V delay, different densities of cardiac fibroblasts will be incorporated into the thin film, which may slow conduction through electronic loading of the excitable cells. Determining the possible range of A-V delays is important for future in vivo experiments, as the desired A-V delay and corresponding AVN-MTF size will be species- (and patient-) specific. The possibility of creating unidirectional conduction block in the AVN-MTF will also be explored to prevent retrograde V-A conduction. This will be achieved with an MTF architecture that creates a source-sink mismatch during retrograde activation. This is important for preventing arrhythmias and maintaining normal sinus rhythm. The effect of electronic loading of the host myocardium on the AVN-MTF will also be explored. This is important as different in vivo transplant conditions may require bridging areas of scarred myocardium, which may affect the conduction characteristics of the bridge.

In describing embodiments of the invention, specific terminology is used for the sake of clarity. For purposes of description, each specific term is intended to at least include all technical and functional equivalents that operate in a similar manner to accomplish a similar purpose. Additionally, in some instances where a particular embodiment of the invention includes a plurality of system elements or method steps, those elements or steps may be replaced with a single element or step; likewise, a single element or step may be replaced with a plurality of elements or steps that serve the same purpose. Further, where parameters for various properties are specified herein for embodiments of the invention, those parameters can be adjusted up or down by 1/20^{th}, 1/10^{th}, ⅕^{th}, ⅓^{rd}, ½, etc., or by rounded-off approximations thereof, unless otherwise specified. Moreover, while this invention has been shown and described with references to particular embodiments thereof, those skilled in the art will understand that various substitutions and alterations in form and details may be made therein without departing from the scope of the invention; further still, other aspects, functions and advantages are also within the scope of the invention. The contents of all references, including patents and patent applications, cited throughout this application are hereby incorporated by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

What is claimed is:

1. A pacemaker, comprising:
a flexible polymer layer; and
an anisotropic tissue structure comprising a population of pacemaker cells coated on the flexible polymer layer, wherein the tissue structure is configured for epicardial or myocardial attachment and is further configured to propagate an action potential through the attached tissue.

2. The pacemaker of claim 1, wherein said cells are selected from the group consisting of sinoatrial node cells, atrioventricular node cells, embryonic stem cells, adult mesenchymal stem cells, committed ventricular progenitor cells, and genetically engineered cells.

3. The pacemaker of claim 1, wherein said cells are human cells.

4. The pacemaker of claim 1, wherein said cells express an ion channel that promotes electrical excitability.

5. The pacemaker of claim 4, wherein said ion channel is encoded by an HCN gene.

6. The pacemaker of claim 5, wherein said HCN gene is a human HCN.

7. A method for producing a pacemaker, comprising
providing a base layer;
depositing a sacrificial polymer on the base layer, thereby generating a sacrificial polymer layer;
depositing a flexible polymer layer that is more flexible than the base layer on the sacrificial polymer layer;
patterning a biopolymer on the flexible polymer layer;
seeding cells on the flexible polymer layer;
culturing the cells such that an anisotropic tissue forms on the flexible polymer layer; and
releasing the flexible polymer layer comprising the anisotropic tissue from the base layer, thereby producing a pacemaker comprising the tissue structure, wherein the tissue structure is configured for epicardial or myocardial attachment and is further configured to propagate an action potential through the attached tissue.

8. The method of claim 7, wherein said cells are selected from the group consisting of a sinoatrial node cells, atrioventricular node cells, embryonic stem cells, adult mesenchymal stem cells, committed ventricular progenitor cells, and genetically engineered cells.

9. The method of claim 7, wherein said cells are human cells.

10. The method of claim 7, wherein said cells express an ion channel that promotes electrical excitability.

11. The method of claim 10, wherein said ion channel is encoded by an HCN gene

12. The method of claim 11, wherein said HCN gene is a human HCN.

13. A method of treating a subject with a bradyarrythmia, comprising:
providing a pacemaker comprising a population of cells coated on a flexible polymer layer, wherein said cells form a tissue structure, wherein the tissue structure is configured for epicardial or myocardial attachment and is further configured to propagate an action potential through the attached tissue; and
attaching said tissue structure to the epicardium or myocardium of said subject.

14. The method of claim 13, wherein said cells are selected from the group consisting of a sinoatrial node cells, atrioventricular node cells, embryonic stem cells, adult mesenchymal stem cells, committed ventricular progenitor cells, and genetically engineered cells.

15. The method of claim 13, wherein said cells are human cells.

16. The method of claim 13, wherein said cells express an ion channel that promotes electrical excitability.

17. The method of claim 16, wherein said ion channel is encoded by an HCN gene.

18. The method of claim 17, wherein said HCN gene is a human HCN.

19. The method of claim 13, wherein the method further comprises administering the pacemaker to the heart tissue by means of a transmyocardial catheter.

20. A method of treating a patient with an AV-node conduction defect, comprising:
providing a pacemaker comprising a population of cells coated on a flexible polymer layer, wherein said cells form a tissue structure, wherein the tissue structure is configured for epicardial, myocardial attachment and is further configured to propagate an action potential through the attached tissue; and
attaching said tissue structure to the epicardium or myocardium of said patient such that the AV-node is bypassed.

21. The method of claim 20, wherein said cells are selected from the group consisting of a sinoatrial node cells, atrioventricular node cells, embryonic stem cells, adult mesenchymal stem cells, committed ventricular progenitor cells, and genetically engineered cells.

22. The method of claim 20, wherein said cells are human cells.

23. The method of claim 20, wherein said cells express an ion channel that promotes electrical excitability.

24. The method of claim 23, wherein said ion channel is encoded by an HCN gene.

25. The method of claim 24, wherein said HCN gene is a human HCN.

26. The method of claim 20, wherein the method further comprises administering the pacemaker to the heart tissue by means of a transmyocardial catheter.
